(19) United States
(12) Patent Application Publication
(10) Pub. No.: US 2005/0100888 A1
(43) Pub. Date: May 12, 2005

(54) Methods based on hormone dependency of primary cancer cells

(75) Inventors: Raoul Charles Coombes, London (GB); Brian Foxwell, Middlesex (GB)

Correspondence Address: NIKOLAI & MERSEREAU, P.A., 900 SECOND AVENUE SOUTH, SUITE 820, MINNEAPOLIS, MN, 55402, US

(73) Assignee: Imperial College Innovations Limited, London (GB)

(21) Appl. No.: 10/932,370

(22) Filed: Aug. 31, 2004

Related U.S. Application Data
(63) Continuation of application No. PCT/GB03/00878, filed on Mar. 3, 2003.

(30) Foreign Application Priority Data
Mar. 2, 2002 (GB) 0204967.4

(51) Int. Cl. C12Q 1/70; C12Q 1/68; G06F 19/00; G01N 33/48; G01N 33/50; C12N 15/867

(52) U.S. Cl. 435/5; 435/6; 435/456; 702/19

(57) ABSTRACT

A method for providing information for deciding on a therapeutic strategy for a patient, comprising the step of assessing the activity of a transcription factor in a cell or cells from the patient, comprising the steps of (1) exposing the cell or cells to a recombinant viral vector comprising a reporter gene which comprises a promoter under the control of the transcription factor (transcription factor-dependent promoter); and (2) assessing the expression of the reporter gene in the said cell or cells. A method for characterising the hormone dependency of a primary cancer cell or cells, comprising the steps of (1) exposing the cell or cells to a recombinant viral vector comprising a reporter gene which comprises a hormone-dependent promoter; and (2) assessing the expression of the reporter gene in the said cell or cells. A method for choosing a treatment regime for a patient with a cancer which may be hormone-dependent, comprising the steps of (1) exposing primary cancer cells from the patient to a recombinant viral vector comprising a reporter gene which comprises a hormone-dependent promoter, (2) determining the response of the reporter gene to exposure of the cell or cells to the hormone and/or other test compound; (3) deciding on a treatment regime making use of the information on the reporter gene behaviour. The cancer is preferably breast cancer or endometrial cancer. The recombinant viral vector is preferable a recombinant adenoviral vector. The method may be used to determine whether the patient should be treated using tamoxifen, an aromatase inhibitor, complete estrogen antagonist or chemotherapy.

The present invention relates to methods for the diagnosis and treatment of human disease, particularly cancer, still more particularly breast cancer, and to reagents useful in such methods.

Transcription factors are proteins that regulate cellular processes by activating and/or repressing the expression of specific genes and thereby the levels of the proteins encoded by regulated genes. Many transcription factors are implicated in human disease, including cancer. Often these diseases are characterised by gain or loss of the activity of a given transcription factor. Examples of gain/loss of transcription factor activities include mutation of the p 53 gene (Nigro et al. 1989 Nature 342: 705-8) and Rb in retinoblastoma (Zheng L, Lee W H. Exp Cell Res. 264: 2-18).

Transcription factors are involved in hormone signalling. Breast cancer is a classic example of a hormone dependent cancer and the hormone estrogen (E2) regulates the growth of the majority of breast cancers. Other examples of hormone dependent cancer include endometrial cancer, ovarian cancer, thyroid cancer and prostate cancer as discussed in the following references: Sherman et al. The Management of Metastatic Differentiated Thyroid Carcinoma. Rev Endocr Metab Disord 2000. 1 (3): 165-71; Makar et al. Hormone Therapy in Epithelial Ovarian Cancer. Endocr Relat Cancer. 7 (2): 85-93; Schroder et al. Endocrine Treatment of Prostate Cancer. BJU Int. 1999. 83: 161-70; Chen et al. Endometrial Cancer. Oncology 1999. 13 (12): 1665-70; Feldman and Feldman. The development of androgen-independent prostate cancer (2001) Nature Reviews Cancer 1: 34-45.

Transcription factors regulate gene expression by binding to specific DNA sequences in the promoters of regulated genes. Their binding stimulates recruitment of the basal transcription factors including RNA polymerase II, resulting in messenger RNA synthesis. Transcription factors stimulate recruitment of the basal transcription factors including RNA polymerase II through direct interaction with the basal transcription factors and/or by recruitment of transcriptional co-regulator complexes, which in turn recruit the RNA polymerase II complexes through direct interaction. In addition, co-regulator complexes facilitate gene expression by chromatin remodelling and include complexes that remodel chromatin by modification of core histones, for example by acetylation, arginine methylation. Transcription repression is brought about by several mechanisms. In the case of nuclear receptors interaction with N-CoR/SMRT results in recruitment of the histone deacetylases complex and consequent chromatin remodelling (Lemon and Tjian (2000) Genes Dev. 14: 2551-2569; Maldonado et al. (1999) Cell 99: 455-458).

Transcription factor activities are routinely studied in established cell lines using reporter genes. These are comprised of a synthetic promoter containing sequences required for basal transcription factor binding (e.g. TATA box and transcription start site), the specific DNA sequences to which the transcription factor of interest binds and sequences encoding a protein, often of bacterial origin, whose activity can be readily assayed. Examples include bacterial chloramphenicol acetyl transferase (CAT) that converts chloramphenicol to acetyl-chloramphenicol, β-galactosidase that can be used in simple colorimetric assays, firefly luciferase and green fluorescent protein (see, for example, Bronstein et al (1994) Chemiluminescent and bioluminescent reporter gene assays Anal Biochem 219, 169-181; Tsien (1998) The green fluorescent protein Ann Rev Biochem 67, 509-544).

Present methods for studying cells from biopsies from tumours rely on methods for the detection of transcription factor levels and more involved methods for the identification of mutations in the genes encoding relevant transcription factors. The ability to monitor the activity of important transcription factors in these disease states would facilitate treatment of these diseases.

In the case of breast cancer the hormone oestrogen (E2) regulates the growth of the majority of breast cancers. E2 action is mediated by the transcription factors oestrogen receptor a (ERα) and estrogen receptor β (ERβ) (Ali and Coombes (2002) Nature Reviews Cancer 2: 101-112).

The majority of patients present with localised disease, also known as primary breast cancer. The usual treatment is surgical excision of the tumour, followed by adjuvant therapy. Adjuvant therapies for ERα-positive disease are designed to reduce oestrogen levels or block its activity by binding to the receptor, as exemplified by tamoxifen. Tamoxifen is now the first line adjuvant treatment for ERα-positive disease in pre- and post-menopausal women and is beneficial in the treatment of metastatic disease, as well as localized disease. However, approximately 30%, of patients with ERα-positive disease do not respond to tamoxifen. Moreover, a substantial proportion of patients presenting with localized disease and all patients presenting with metastatic disease that initially respond to tamoxifen treatment become resistant. In the case of patients who initially respond but become resistant to tamoxifen ERα expression is lost in only ˜10% of cases. Moreover, one-third of resistant patients show a clinical response to treatment with a different anti-estrogen such as Faslodex (ICI 182, 780) or the use of aromatase inhibitors, drugs that inhibit estrogen synthesis. Nevertheless and despite issues of de novo or acquired resistance to tamoxifen, it has become the adjuvant agent of first choice (Ali and Coombes (2002) Nature Rev. Cancer 2: 101-112).

ERα is a member of the nuclear receptor superfamily of transcription factors that activates gene expression upon binding as homodimers to small palindromic sequences known as estrogen response elements (EREs) in promoters of estrogen responsive genes. EREs conform to the general sequence GGTCAnnnTGACC, with some diversity from this sequence still providing a functional ERE (Klinge (2001) Nucleic Acids Res. 29: 2905-2919). Transcription activation by ERα requires estrogen binding to the ligand or hormone binding domain (Shiau et al. (1998) Cell 95: 927-937) and is mediated by co-regulator recruitment upon ligand binding (Glass & Rosenfeld (2000) Gene & Dev. 14: 121-141).

Resistance to endocrine therapy could theoretically arise through mutation of the ERα gene resulting in ERα protein that is inactive, is active in a ligand-independent manner or is activated by tamoxifen. Some ERα mutations have been described (Hopp and Fuqua S A. (1998) J Mammary Gland Biol Neoplasia. 3: 73-83). Resistance could also arise by growth factor-stimulated post-translational modification of ERα that results in an increase in ERα activity such that ERα is activated in the absence of ligand, or is activated by suboptimal concentrations of E2 and/or is activated, rather than inhibited by tamoxifen (Chen et al. (2001) Mol. Cell. 6, 127-137). Mutation and/or reduction in levels of co-repressor complexes and/or increased levels of co-activator proteins could also lead to resistance. In vitro resistance to tamoxifen has been correlated with reduction in amounts of the co-repressor N-CoR (Lavinsky et al. PNAS 95: 2920-2925), whilst overexpression and/or amplification of the AIB1 coactivator is observed in almost 5% of breast cancers (Anzick et al (1997) Science 277: 965-968; Bautista et al. (1998) Clin. Cancer Res. 4: 2925-2929). See also Kothari (2003) Br J Cancer in press.

At present the likelihood of response to endocrine therapies is determined by immunohistochemical detection of ERα. If a tumour is deemed to be ERα-positive then adjuvant tamoxifen treatment for 5 years is used. However, immunohistochemical detection or oestrogen binding assay are uninformative as to the activity status of ERα. It would, however, be highly desirable in ERα-positive tumours to be able to characterise the ERα activity.

Reporter genes containing promoters derived from those of an oestrogen-responsive gene such as the pS2 (Berry et al. (1989) Proc Natl Acad Sci U S A. 86: 1218-1222) or the xenopus vitelogenin (Klein-Hitpass et al. (1988) Nucleic Acids Res. 16: 647-663) genes or those containing one or several oestrogen response elements (EREs) in tandem have been used extensively to study ERα activity in breast cancer-derived cell lines such as MCF-7 cells or in ERα-negative cell lines in which ERα has been introduced.

We have developed a novel functional assay based on a transcription factor reporter gene-(for example a reporter gene under the control of a hormone response element) in which purified primary cells from a patient, for example cancer cells, maintained in short term cultures, can be infected with the reporter. We show that using this assay, transcription factor, for example hormone receptor, activity can be assessed reliably, in vitro, in primary cells, for example primary cancer cells. The information obtained can be used in guiding treatment of the patient. We provide methods for treating patients, based on alterations to transcription factor/hormone receptor function identified using the functional assay.

In particular, we have developed methods for the purification of breast cancer cells from primary tumours as well as those obtained from secondary sites such as bone marrow, pleural effusions and ascites and have defined conditions for their maintenance in culture for several weeks. Using these cells we show that ER activity can be assessed reliably in vitro, in cancer cells from primary tumours and in metastatic cancer cells. The information thus obtained can be used in guiding treatment of the patient. The assay may be used to determine (a) whether ERα is inactive, in which case chemotherapy may be advisable, (b) whether ERα activity is not inhibited by tamoxifen, in which case the use of a drug which lowers oestrogen levels, for example aromatase inhibitors (and/or LHRH agonists such as Goserelin, Zoladex or Prostap in premenopausal patients), or an antioestrogen such as Faslodex may be indicated, or (c) is active in the absence of ligand in which case the use of Faslodex or chemotherapy might be indicated.

A first aspect of the invention provides a method for providing information for deciding on a therapeutic strategy for a patient, comprising the step of assessing the activity of a transcription factor in a cell or cells from the patient, comprising the steps of (1) exposing the cell or cells to a recombinant viral vector comprising a reporter gene which comprises a promoter under the control of the transcription factor; and (2) assessing the expression of the reporter gene in the said cell or cells. The method may comprise the step of determining the response of the reporter gene to exposure of the cell or cells to a test compound or environment.

The information regarding the activity of the transcription factor is used in deciding on a therapeutic strategy for the patient. For example, the information may be used in deciding whether the patient has a particular disease or condition, or in further characterising the form of the disease or condition that the patient has. The therapeutic strategy may be a prophylactic strategy, for example if the patient has no (for example if there is a family history of a particular disease or condition) or preliminary symptoms. By therapeutic strategy or regime is included a strategy concerning, for example, surgery, administration of pharmaceutical compounds and/or diet.

Transcription factors to which the reporter gene may be designed to respond are discussed above and further below, and include NF-κB, ETS1, the tumour suppressor gene p53, c-myc, a Forkhead transcription factor, E2F, AP1 and SRE.

In a preferred embodiment the transcription factor is a nuclear hormone receptor protein and the reporter gene comprises a response element for the nuclear hormone receptor protein. Thus, the reporter gene preferably comprises a hormone-dependent promoter.

Thus, a second aspect of the invention provides a method for characterising the hormone dependency of a primary cancer cell or cells, comprising the steps of (1) exposing the cell or cells to a recombinant viral vector comprising a reporter gene which comprises a hormone-dependent promoter; and (2) assessing the expression of the reporter gene in the said cell or cells.

By a “primary cancer cell” is meant a cancer cell derived directly from a patient, for example from a primary or secondary tumour. The term encompasses a cell of a short term culture (starting from a cell or cells removed from a patient), for example after culture for a limited period, for example of up to 20 weeks, but does not include a cell of an immortalised cell line.

It may also be useful to assess the activity of a transcription factor, or to characterise the hormone dependency of primary non-cancerous cells from a patient. For example, analysis of normal primary breast epithelial cells may also be informative, on its own or alongside analysis of cancer cells.

Using the methods of the invention, we have found that it is possible to obtain reliable results when working on clinical samples (ie when only a very small number (for example about 10000-100000) of cells, for example cancer cells, are available for analysis), for example in relation to breast cancer.

Preferably the hormone-dependent promoter is an estrogen-dependent promoter. Thus, the promoter preferably comprises one or more Estrogen Response Elements (EREs), for example two EREs. EREs are foung in estrogen-regulated genes. Still more preferably, the Estrogen Response Element is the Estrogen Response Element derived from the progesterone receptor (PR) gene or the PS2 (trefoil related protein) gene. The ERE has several different sequences that may be used in the construct (Klinge et al. Estrogen Receptor Interaction with estrogen response elements. Nucleic Acids Res 2001. 29(14): 2905-19).

Alternatively, the hormone-dependent promoter may be an androgen-dependent promoter (ie comprising one or more Androgen Response Elements (AREs)) or a thyroid hormone dependent promoter (ie comprising a Thyroid hormone Response Element (TRE) or a progesterone receptor dependent promoter or other steroid hormone dependent promoter or retinoic acid receptor dependent promoter or PPAR (peroxisome proliferator receptor) or VDR (vitamin D receptor) dependent promoter.

Thus, the hormone-dependent promoter comprises a binding site for a nuclear receptor DNA binding protein such as a steroid hormone receptor protein, as well known to those skilled in the art. The nuclear receptor DNA binding protein superfamily includes estrogen receptor (ER), androgen receptor (AR), progesterone receptor (PR), retinoic acid receptor (RAR), peroxisome-proliferator activated receptors (PPAR), thyroid hormone receptor and the like (see Mangelsdorf et al (1995) Cell 83, 835-839 for a review and nomenclature; also Chawla et al (2001) Nuclear receptors and lipid physiology: opening the X-files Science 294 (5548), 1866-1870).

Preferably the primary cancer cell is a primary breast cancer cell or other potentially estrogen-dependent primary cancer cell. Thus, the cell may be an endometrial cancer (EC) cell or ovarian cancer cell. At least two different types of EC are thought to exist (see Emons et al (2000) Endocr Relat Cancer 7 (4), 227-242). Type I is associated with an estrogen-dominated hormonal environment, endometrioid histology and development from endometrial hyperplasia; this form is considered to be responsive to endocrine therapy. Type I is not associated with an estrogen-dominated hormonal environment, has a serous histology and develops from atrophic endometrium in elderly women; this form is not considered to be responsive to endocrine therapy. The method of the invention may be most informative in relation to Type I EC but may also be useful in assessing cells of Type II EC.

Most preferably, the cell is a primary breast cancer cell. The method of the invention may be particularly useful in relation to breast cancer because of the pattern of progression typically seen in breast cancer (in which there may be more than one change in the hormone responsiveness of the cancer), and the range of treatments available (for example, several endocrine therapy options; chemotherapy; and surgery). We have demonstrated that the method may be used reliably in a clinical setting in relation to primary breast cancer cells.

The method of the invention may be less useful in relation to other cancers (for example prostate cancer) in which the therapeutic options are more limited. This is because surgery is considered to be the only effective option for prostate cancer if endocrine therapy fails.

As noted above, the reporter gene comprises a hormone-dependent promoter. Thus, transcription of the reporter gene is considered to be under the control of the hormone-dependent promoter. The transcribed nucleic acid may be any nucleic acid whose transcription is detectable, for example leads to the production of a detectable product.

The detectable product may be the transcribed RNA (including a portion or processed version thereof); this may be detected by techniques well known to those skilled in the art, for example a technique involving hybridisation of the transcribed RNA (or portion thereof) to one or more complementary nucleic acids. For example, the transcribed RNA may be detected by a technique involving the polymerase chain reaction (PCR) or other amplification reaction; preferably the technique involves quantitative reverse-transcriptase PCR (RT-PCR). Suitable techniques may be described in, for example, Van Trappen P O et al. Molecular quantification and mapping of lymph-node micrometastases in cervical cancer. Lancet 2001 Jan. 6; 357(9249): 15-20, Aerts J et al. A real-time quantitative reverse transcriptase polymerase chain reaction (RT-PCR) to detect breast carcinoma cells in peripheral blood. Ann Oncol. 2001 January; 12 (1): 39-46.

Alternatively or additionally, the transcribed RNA may encode a polypeptide which may be detected immunologically or as a result of its enzymatic or other biological activity. For example, the transcribed RNA may encode an enzyme such as β-galactosidase which is capable of producing a colour change (or fluorescence change) in a reagent (for example IPTG or CRPG (chloro-phenol-red-guanothiocyanate)).

The transcribed RNA may encode a luciferase enzyme (for example from firefly (Photinus pyralis) or seapansy (Renilla reiformis)). Luciferase catalyses a reaction involving D-luciferin which results in light emission, as well known to those skilled in the art. Alternatively, the transcribed RNA may encode a fluorescent protein, for example a protein belonging to the Green Fluorescent Protein (GFP) family (for example the Green Fluorescent protein from Aqueorea Victoria). GFPs are intrinsically fluorescent proteins, which may emit blue, yellow light or green light. Miyawaki et al (1997) Nature 388, 882-887 describes a GFP-based Ca²⁺ sensing system; Mitra et al (1996) Gene 173, 13-17 describes a two-GFP-based system for use in identifying protease inhibitors; WO 97/28261 discloses a two-GFP system in which the GFP donor and GFP acceptor are linked by a peptide containing a protease cleavage site. WO 95/07463 describes uses of GFPs; WO 96/23898 relates to a method of detecting biologically active substances using GFPs; Heim & Tsien (1996) Current Biology 6, 178-182 relates to engineered GFPs with improved brightness, longer wavelengths and fluorescence resonance energy transfer (FRET); Poppenborg et al (1997) J. Biotechnol. 58, 79-88 relates to GFPs as a reporter for bioprocess monitoring; Park & Raines (1997) Protein Science 6, 2344-2349 relates to a GFP as a signal for protein-protein interactions; Niswender et al (1995) J. Microscopy 180, 109-116 relates to quantitative imaging of GFP in cultured cells; Chalfie et al (1994) Science 263, 802-805 relates to GFP as a marker for gene expression; Hampton et al (1996) Proc. Natl. Acad. Sci. USA 93, 828-833 relates to the in vivo examination of membrane protein localisation and degradation with GFP; Heim et al (1995) Nature 373, 663-664 relates to mutant GFPs with altered fluorescent properties; Mosser et al (1997) BioTechniques 22, 150-161 relates to the use of a dicistronic expression cassette encoding GFP for the screening and selection of cells expressing inducible gene products; Suarez et al (1997) Gene 196, 69-74 relates to GFP-based reporter systems for genetic analysis of bacteria; Niedenthal et al (1996) Yeast 12, 773-778 relates to GFP as a marker for gene expression and subcellular localisation in budding yeast; Mahajan et al (1998) Nature Biotech. 16, 547-552 relates to the probing of Bcl-2 and Bax interactions in mitochondria using GFPs and FRET; and Prescott et al (1997) FEBS Lett 411, 97-101 relates to the use of GFP as a marker for assembled mitochondrial ATP synthase in yeast. GFPs and their uses have been reviewed in Pozzan et al (1997) Nature 388, 8340-835, Misteli & Spector (1997) Nature Biotechnology 15, 961-964; and Cubitt et al (1995) Trends Biochem. Sci. 20, 448-455.

GFPs may be particularly useful in that more than one reporter gene may be introduced into the cell (as discussed further below), each expressing a GFP with a different wavelength of excitation and/or emission, so that the output of each reporter gene may be distinguished.

As a further alternative the reporter gene may encode chloramphenicol acetyltransferase (CAT), as well known to those skilled in the art. CAT catalyses the transfer of the acetyl group from acetyl-CoA to choramphenicol. CAT may be quantified immunologically, for example using an ELISA assay, as well known to those skilled in the art. Alternatively, it may be quantified by measuring enzymic activity, for example by following the appearance of acetylated chloramphenicol (for example using radioactively labelled choramphenicol (for example [¹⁴C]choramphenicol) and physical separation means (for example thin layer chromatography or organic extraction)). The reporter gene may alternatively encode secreted alkaline phosphatase (SEAP), which can be tested using medium from infected cells (Cullen & Malim (1992) Meth. Enzymol. 216: 362-368; Kain (1996) Use of secreted alkaline phosphatase as a reporter of gene expression in mammalian cells. Methods in Molecular Biology vol 63 Humana Press Totowa N.J.). The reporter gene may alternatively encode a tagged protein, for instance with a 6-His tag that my be detected using non-immunological methods.

Methods and apparatus for detecting and/or quantifying reporter gene products or their enzymic products will be well known to those skilled in the art, and may include measurements using a spectrophotometer, fluorometer or luminometer. Other suitable methods, systems or instruments (depending on the reporter gene, as will be clear to the skilled person) include Light-Cycler®, Taqman®, histology, microscopy, radiography or LCM.

The term “viral vector” will be well known to those skilled in the art. It is particularly preferred that the viral vector is an adenoviral vector, though it may alternatively be a lentiviral vector. It encompasses any viral vector, for example adenoviral vector or lentiviral vector that is suitable for introducing recombinant nucleic acid into a eukaryotic cell, preferably a human cell. HSV, AAV, vaccinia and parvovirus vectors may also be suitable.

Preferred lentiviral vectors include those described in Verma & Somia (1997) Nature 389, 239-242. Preferred vectors include lentivirus vectors and adenoviral vectors, for example vectors similar to those described in Foxwell et al (2000) Ann Rheum Dis 59 Suppl 1, 154-59 or Bondeson et al (2000) J. Rheumatol 27 (9), 2078-2089.

The adenoviral vector (for example) may be any serotype of adenovirus but is preferably one that is capable of infecting or otherwise introducing recombinant nucleic acid into a human cell. Preferably it is of serotype 5 (see, for example, Shenk (1996) Fields Virology Fields et al Eds (Lippincott, Philadelphia), pp 2111-2148; Horwitz (1996) Fields Virology Fields et al Eds (Lippincott, Philadelphia), pp 22149-2171. It may comprise a complete adenoviral virion, consisting of a core of nucleic acid and a protein capsid. Alternatively, it may comprise a naked adenoviral genome or a protein capsid with a minimal adenoviral genome comprising the non-adenoviral nucleic acid and adenoviral packaging signal but with most of the adenoviral genome deleted; this may be termed a “gutless” virus.

The viral vector, for example adenovirus vector to which the primary cancer cell or cells is exposed is preferably replication-deficient. Otherwise, it is likely to replicate and kill the cell.

It is particularly preferred that the adenoviral vector is an adenoviral vector as described in He et al (1998) PNAS 95, 2509-2514. Characteristics of adenoviruses and adenoviral vectors are described in this reference and in references cited therein. The recombinant adenoviral vector to which the primary cancer cell or cells is exposed may be prepared using a method based on that described in He et al (1998), for example as described in Example 1. This method may allow incorporation of up to about 10 kb of reporter gene sequence; multiple reporter genes may be incorporated into one adenoviral vector construct. This method makes use of recombination between the adenoviral genome (which may be modified; for example to render it replication-deficient) and nucleic acid comprising the reporter gene sequence (for example comprising an lacZ coding region under the control of an ERE), for example a plasmid comprising the reporter gene.

The adenoviral vector may comprise a non-hormone dependent reporter gene (for example capable of expressing a GFP), for example a reporter gene under the control of a constitutive or unregulated promoter such as the CMV promoter. This may be useful in monitoring the level of cellular infection achieved. However, as discussed in Example 1, the presence of such a reporter gene may lead to a higher background level of expression of the hormone-regulated reporter gene (and/or lower induced level of expression of the hormone-regulated reporter gene) and may therefore not always be desirable.

Other methods of delivery include adenoviruses carrying external DNA via an antibody-polylysine bridge (see Curiel Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with the DNA construct or other genetic construct of the invention, wherein the antibody is specific for either wild-type adenovirus or a variant adenovirus in which a new epitope has been introduced which binds the antibody. The polycation moiety binds the DNA via electrostatic interactions with the phosphate backbone. The adenovirus, because it contains unaltered fibre and penton proteins, is internalised into the cell and carries into the cell with it the DNA construct of the invention. It is preferred if the polycation is polylysine.

The DNA may also be delivered by adenovirus wherein it is present within the adenovirus particle, for example, as described below.

High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle.

Alternative targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein. Mutant adenoviruses which replicate selectively in p53-deficient human tumour cells, such as those described in Bischoff et al (1996) Science 274, 373-376 are also useful for delivering the genetic construct of the invention to a cell.

The recombinant viral, for example adenoviral, vector may comprise more than one transcription factor-controlled reporter gene. For example, breast cancer is characterised by increased proliferation and decreased apoptosis as well as increased invasiveness of luminal epithelial cells. The recombinant viral vector may comprise reporter genes suitable for providing information on each of these properties, in order to assist clinicians with devising suitable strategies to combat these adverse properties. Alternatively, the primary cancer cell or cells may be exposed to more than one recombinant viral vector, for example one comprising a hormone-dependent reporter gene and the second or further vectors comprising the further reporter genes relating to invasion, apoptosis or proliferation.

The hormone-dependent promoter-controlled reporter gene provides a read out of the function of the hormone receptor, for example ER, and is useful as a guide to selecting suitable agents for antagonising the ER, thereby inhibiting proliferation induced via this transcription factor. The technique can be extended to assist in identifying signalling via other transcription factors.

Two transcription factors NF-κB and ETS1 are important in activating expression of genes whose products are involved in invasion through the basement membrane (Huang et al, Oncogene, 20, 4188 (2001); Oikawa et al, BBRC, 289, 39 (2001). Both bind to defined DNA response elements which can be incorporated as reporter genes with the adenovirus. An NF-κB reporter system is described in Sanlioglu et al (2001) J. Biol Chem 276, 30188. Patients whose tumour cells show activity of these 2 transcription factors should benefit from therapy with Metalloproteinase Inhibitors.

The tumour suppressor gene p53 is essential in cellular response to DNA damage and other stresses. The p53 gene is often mutated in cancers, including breast cancer and monitoring its activity would be an important indicator in the apoptotic response of cancer cells. The c-myc proto-oncogene also plays an important role in apoptotic responses. Furthermore, the transcription factor Forkhead is also involved in apoptotic responses. Failure to undergo apoptosis is a hallmark of certain types of cancers. Apoptosis is normally triggered in abnormal cells by p53 and c-inyc. Response elements to these factors may be useful in assessing apoptosis potential, and may therefore be included in the recombinant adenoviral vector. Several new therapies are being developed which aim to enhance apoptosis. These include BH3 analogues, designed to mimic pro-apoptotic bcl-2 interactants. (Degterev et al. Identification of small-molecule inhibitors. Nat Cell Biol 2001. 3 (2): 173-82).

E2F, AP1, SRE transcription factor activities are often stimulated in response to proliferative signals. Tumour cells with active E₂F/Rb, SRE and/or AP1 activity are likely to be more amenable to treatment with anti proliferation agents, e.g., cytotoxics. Reporter genes with promoters regulated by such transcription factors may therefore also be included in the recombinant adenoviral vector.

The primary cancer cell or cells which are exposed to the recombinant viral, preferably adenoviral, vector are preferably purified cancer cells, ie separated from non-cancerous cells. Similarly, it is preferred that other purified cell types (ie separated from other cell types which are not of interest) are used, as appropriate. Methods by which primary breast cancer cells may be prepared and cultured are described in the Examples. Suitably, the methods may involve immunolabelled magnetic bead separation techniques, as known to those skilled in the art, particularly as described in Example 3. It is preferred that at least 80 or 85%, more preferably 90, 95, 96, 97, 98 or 99% of the cells to which the viral vector is exposed are cancer cells as opposed to non-cancerous cells, as discussed in Example 3.

A further aspect of the invention provides the use of a primary cancer cell or cells (preferably breast cancer cell or cells) purified using immunolabelled magnetic beads, or cells obtained in a fine needle aspirate, in a method for characterising the hormone dependency or other transcription factor activity of the cells using a reporter gene which comprises a promoter dependent on the hormone or other transcription factor. The method may be used for determining a treatment strategy for the patient from whom the cell or cells are derived. Preferences are as indicated above in relation to the methods of the invention.

Analysis may be performed on a single cell, but it is preferred that several cells are analysed (either individually or as a group) and the results pooled. This may mean that the results obtained are more representative of the cancer or tumour than may be results obtained from a single cell.

Methods by which single cells may be analysed include methods in which the technique of Laser Capture Microdissection (LCM) is used. This technique may be used to collect single cells or homogeneous cell populations for molecular analysis and is described in, for example, Jin et al (1999) Lab Invest 79 (4), 511-512; Simone et al (1998) Trends Genet 14 (7), 272-276; Luo et al (1999) Nature Med 5 (1), 117-122; Arcuturs Updates, for example June 1999 and February 1999; U.S. Pat. No. 5,859,699 (all incorporated herein by reference). The cells of interest are visualised, for example by immunohistochemical methods, and transferred to a polymer film that is activated by laser pulses. Microscopes useful in performing LCM are manufactured by Arcturus Engineering, Inc., 1220 Terra Bella Avenue, Mountain View, Calif. 94042, USA.

LCM may be used with other isolation/detection methods. For example, LCM may be used following an isolation/detection method which enriches the sample for the target cell type. LCM may be particularly useful in the analysis of primary cancer cells obtained from blood.

Because cells may not be alive following LCM, it may be most appropriate to use LCM in the analysis of cells following introduction of the reporter gene into the cells rather than prior to introduction of the reporter gene into the cells.

It will be appreciated that it may be desirable to expose the recombinant viral vector, for example adenovirus, to cell line cells, for example cancerous cell line cells, for example in order to determine suitable conditions for cell infection and reporter gene assays.

A further aspect of the invention provides a method for choosing a treatment regime for a patient with a cancer which may be hormone-dependent, comprising the steps of (1) exposing primary cancer cells from the patient to a recombinant viral, for example adenoviral, vector comprising a reporter gene which comprises a hormone-dependent promoter; (2) determining the response of the reporter gene to exposure of the cell or cells to the hormone and/or other test compound or environment; (3) deciding on a treatment regime making use of the information on the reporter gene behaviour. The test compound or environment may be an antagonist or agonist for the hormone receptor, or the absence of such an antagonist or agonist, as discussed further below.

Thus, the invention provides a method for choosing a treatment regime for a patient with breast or endometrial or ovarian cancer (preferably breast cancer), comprising the steps of (1) exposing primary cancer cell or cells from the patient to a recombinant viral, for example adenoviral, vector comprising an ERE-controlled reporter gene; (2) determining the response of the reporter gene to exposure of the cell or cells to estrogen and/or a test compound or environment; (3) deciding on a treatment regime making use of the information on the reporter gene behaviour. The test compound may be an anti-estrogen compound, for example tamoxifen or Faslodex.

The purpose of ERE-controlled reporter gene is to clarify the molecular mechanisms underlying resistance to endocrine therapy in breast cancer. This is of crucial importance since adjuvant endocrine and chemotherapy can cure a subset of patients with breast cancer. However, a large proportion of patients with ER positive cells relapse, or, in the context of metastatic disease, fail to respond, indicating that they have received sub-optimal treatment, most likely due to deregulated ER function. The adenovirus-delivered ERE-controlled reporter gene assay indicates that a subpopulation of patients express a form of ER that is deficient in transcriptional activity.

We have surprisingly found that in some patients' cells the ERE-controlled reporter gene is activated by tamoxifen. This indicates that treatment of such patients with tamoxifen may be harmful. Accordingly, if the reporter gene is activated by tamoxifen, the patient is not administered tamoxifen, or administration of tamoxifen is terminated. The patient may be administered a drug (or combination of drugs) which lowers oestrogen levels, for example an aromatase inhibitor. These inhibitors stop estrogen from being synthesised and therefore prevent replacement of the tamoxifen bound to the ER by estrogen. A premenopausal patient may be administered instead or (more preferably) in addition an LHRH agonist, for example Goserelin, Zoladex or Prostap, since aromatase inhibitors may fail completely to suppress ovarian oestrogen synthesis.

As will be well known to those skilled in the art, examples of aromatase inhibitors include Formestane (4-OH androstenedione), Exemestane (steroidal, irriversible (type 1) inhibitors), Anastrozole (Arimidex) and Letroxole (non-steroidal, reversible (type 2) inhibitors). It may be useful to use inhibitors of both types in some patients (Harper-Wynne & Coombes (1999) Eur J. Cancer 35, 744-746 Anastrozole shows evidence of activity in postmenopausal patients who have responded or stabilised on formestane therapy). It may also be useful to administer biphosphonates and/or calcium supplements in order to address the risk of osteoporosis, fracture or other musculoskeletal disorders.

We have also surprisingly found that in some patients' cells the ERE-controlled reporter gene is active in the absence of estrogen or tamoxifen. It is considered that epidermal growth factor (EGF) may be driving reporter gene expression. Accordingly, if the reporter gene is active in the absence of estrogen or tamoxifen, then the patient is administered an epidermal growth factor receptor (EGFR) antagonist, or other signalling inhibitor, for example an inhibitor of ErbB2 or MEK signalling. An example of an EGFR antagonist is Iressa (ZD1839; AstraZeneca).

In a third group of patients, a pure antiestrogen (for example Faslodex (ICI 182,780; AstraZeneca), idoxifene (see Coombes et al (1995) Cancer Res 55, 1070-1074), raloxifene or EM-652 (Labrie, F et al, J. Steroid Biochem Mol Biol 79, 213 (2001)) has a stronger inhibitory effect on the reporter gene activity than tamoxifen. Accordingly, if the reporter gene is suppressed more by an agent such as Faslodex than by Tamoxifen, then the patient is administered the agent (for example Faslodex) in place of Tamoxifen.

In a further group of patients, the reporter gene is inactive in the presence of estrogen. For these patients, chemotherapy or surgery may be the preferred treatment, depending on the stage of the disease. For example, when metastasis has occurred, chemotherapy may be the preferred treatment, for example with EM-652 (Pelletier et al, 2001; Neuroendocrinology; 74, 367-74).

A further aspect of the invention provides a recombinant viral, preferably adenoviral, vector comprising a reporter gene which comprises a hormone-dependent promoter for use in a method for choosing a treatment regime for a patient with a cancer which may be hormone-dependent, comprising the steps of (1) exposing primary cancer cells from the patient to the recombinant viral vector; (2) determining the response of the reporter gene to exposure of the cell or cells to the hormone and/or other test compound; (3) deciding on a treatment regime making use of the information on the reporter gene behaviour. The test compound may be an antagonist or agonist for the hormone receptor.

Thus, the invention provides a recombinant viral, preferably adenoviral, vector comprising an ERE-controlled reporter gene for use in a method for choosing a treatment regime for a patient with breast or endometrial or ovarian cancer (preferably breast cancer), comprising the steps of (1) exposing primary cancer cells from the patient to the recombinant viral vector; (2) determining the response of the reporter gene to estrogen and/or a test compound or environment; (3) deciding on a treatment regime making use of the information on the reporter gene behaviour. The test compound may be an anti-estrogen compound, for example tamoxifen or Faslodex.

A further aspect of the invention provides tamoxifen for use in treating a patient with breast cancer or endometrial or ovarian cancer, wherein the patient's cancer has been characterised by a method for choosing a treatment regime, comprising the steps of (1) exposing primary cancer cell or cells from the patient to a recombinant viral, preferably adenoviral, vector comprising an ERE-controlled reporter gene; (2) determining the response of the reporter gene to exposure of the cell or cells to tamoxifen or 4-OHT and/or estrogen and/or an antiestrogen or absence of estrogen; and wherein expression of the ERE-controlled reporter gene is inhibited by tamoxifen or 4-OHT.

A further aspect of the invention provides an aromatase inhibitor and/or LHRH agonist for use in treating a patient with breast cancer or endometrial or ovarian cancer, wherein the patient's cancer has been characterised by a method for choosing a treatment regime, comprising the steps of (1) exposing primary cancer cell or cells from the patient to a recombinant viral, preferably adenoviral, vector comprising an ERE-controlled reporter gene; (2) determining the response of the reporter gene to exposure of the cell or cells to tamoxifen or 4-OHT and/or estrogen and/or an antiestrogen or absence of estrogen; and wherein expression of the ERE-controlled reporter gene is activated by tamoxifen or 4-OHT; and/or determining the response of the reporter gene to exposure of the cell or cells to a medium in which estrogen is absent and wherein expression of the ERE-controlled reporter gene is not activated. This latter test mimics the clinical situation where the patient receives an aromatase inhibitor (or (in premenopausal women) LH2H agonist, particularly alongside an aromatase inhibitor).

A further aspect of the invention provides an epidermal growth factor receptor antagonist (for example Iressa (AstraZeneca)) or c-erb B2 anatagonist (eg Herceptin) or an inhibitor of the EGFR signalling cascade (e.g. MEK inhibitors such as PD98059) for use in treating a patient with breast cancer or endometrial or ovarian cancer, wherein the patient's cancer has been characterised by a method for choosing a treatment regime, comprising the steps of (1) exposing primary cancer cell or cells from the patient to a recombinant viral, preferably adenoviral, vector comprising an ERE-controlled reporter gene; (2) determining the response of the reporter gene to exposure of the cell or cells to tamoxifen or 4-OHT and/or estrogen and/or an antiestrogen; and wherein expression of the ERE-controlled reporter gene is active in the absence of estrogen or tamoxifen.

A further aspect of the invention provides an antiestrogen (for example Faslodex) for use in treating a patient with breast cancer or endometrial cancer or ovarian cancer, wherein the patient's cancer has been characterised by a method for choosing a treatment regime, comprising the steps of (1) exposing primary cancer cell or cells from the patient to a recombinant viral, preferably adenoviral, vector comprising an ERE-controlled reporter gene; (2) determining the response of the reporter gene to exposure of the cell or cells to tamoxifen or 4-OHT and/or estrogen and/or an antiestrogen; and wherein expression of the ERE-controlled reporter gene is inhibited more strongly by then antiestrogen (for example Faslodex) than by tamoxifen or 4-OHT (preferably is inhibited by the anti-estrogen but not by 4-OHT or tamoxifen).

A further aspect of the invention provides a chemotherapeutic agent such as an anthracycline or taxane for use in treating a patient with breast cancer or endometrial cancer, wherein the patient's cancer has been characterised by a method for choosing a treatment regime, comprising the steps of (1) exposing primary cancer cell or cells from the patient to a recombinant viral, preferably adenoviral, vector comprising an ERE-controlled reporter gene; (2) determining the response of the reporter gene to exposure of the cell or cells to tamoxifen or 4-OHT and/or estrogen and/or an antiestrogen; and wherein expression of the ERE-controlled reporter gene is not activated by estrogen. The reporter gene is typically inactive in all test situations.

Expression of the hormone-dependent reporter gene in response to various agents may be judged by reference to expression of a control gene, or may be judged by reference to expression in the absence of the hormone (for example estrogen), for example as described in Example 1. A change of expression level by a factor of at least 2, more preferably 3, 4, 5, 6, 8 or 10 may be considered a significant activation or inhibition, as appropriate.

The behaviour of isolated primary breast cancer cells in tissue culture (for example in response to estrogen), as well as other clinical factors (for example as discussed above) may also be used in deciding on a treatment regime, for example as described in Example 3. The response obtained with further reporter genes (for example relating to invasion, apoptosis or proliferation) may also be considered.

A further aspect of the invention provides a recombinant viral, preferably adenoviral, vector comprising a reporter gene which comprises a hormone-dependent promoter. Preferences for the hormone dependent promoter and reporter gene are as discussed above. In particular, the invention provides a recombinant viral, preferably adenoviral, vector comprising a reporter gene which comprises an estrogen response element (ERE) (or an androgen response element or a thyroid hormone response element). Preferably the reporter gene comprises two EREs. The invention also encompasses a vector (for example pAdTack or pShuttle vector or vector derived therefrom) for preparing the recombinant adenoviral vector of the invention, comprising the reporter gene.

The recombinant viral, preferably adenoviral, vector of the invention (or vector for preparing such a viral, preferably adenoviral, vector) may comprise further reporter genes, as discussed above. However, in a preferred embodiment the adenoviral vector does not comprise a reporter gene under the control of a constitutive promoter, for example a GFP-expressing reporter gene. It is preferred that the reporter gene comprising the hormone-dependent promoter exhibits hormone-dependent transcription (ie an increase in transcription (preferably of at least 2-fold, still more preferably at least 3, 4 or 5-fold)), for example relative to a marker plasmid (for example GTK-CAT plasmid) in MCF7 cells transfected using the calcium phosphate method, as described in Example 1.

A further aspect of the invention provides a recombinant viral, preferably adenoviral, vector of the invention for use in medicine. A still further aspect of the invention provides a pharmaceutical preparation comprising a recombinant viral, preferably adenoviral, vector of the invention and a pharmaceutically acceptable diluent or carrier. This may be, for example, sterile water or saline. The recombinant viral, preferably adenoviral vector may be useful for gene therapy or for monitoring the patient's response, for example to therapy. For example, a luciferase reporter may be used in patients to monitor their response.

A further aspect of the invention provides a kit of parts comprising a recombinant adenoviral vector of the invention and the hormone on which the hormone-dependent promoter is dependent (or an analogue thereof which is able to promote transcription from the reporter ie an agonist of the hormone receptor), and optionally also an antagonist of the hormone receptor and/or a partial antagonist of the hormone receptor. Thus, in a preferred embodiment, the invention provides a recombinant adenoviral vector comprising an estrogen-dependent reporter gene, estrogen (preferably 17β-estradiol; E2) and optionally Tamoxifen (preferably as 4 OHT (4-hydroxytamoxifen)) and/or Faslodex (ICI 182,780) or other antiestrogen. The kit may also comprise a substrate for a reporter gene, if appropriate, for example a substrate for β-galactosidase.

All patent or other documents cited herein are hereby incorporated by reference.

The invention is now described further by reference to the following non-limiting Figures and Examples.

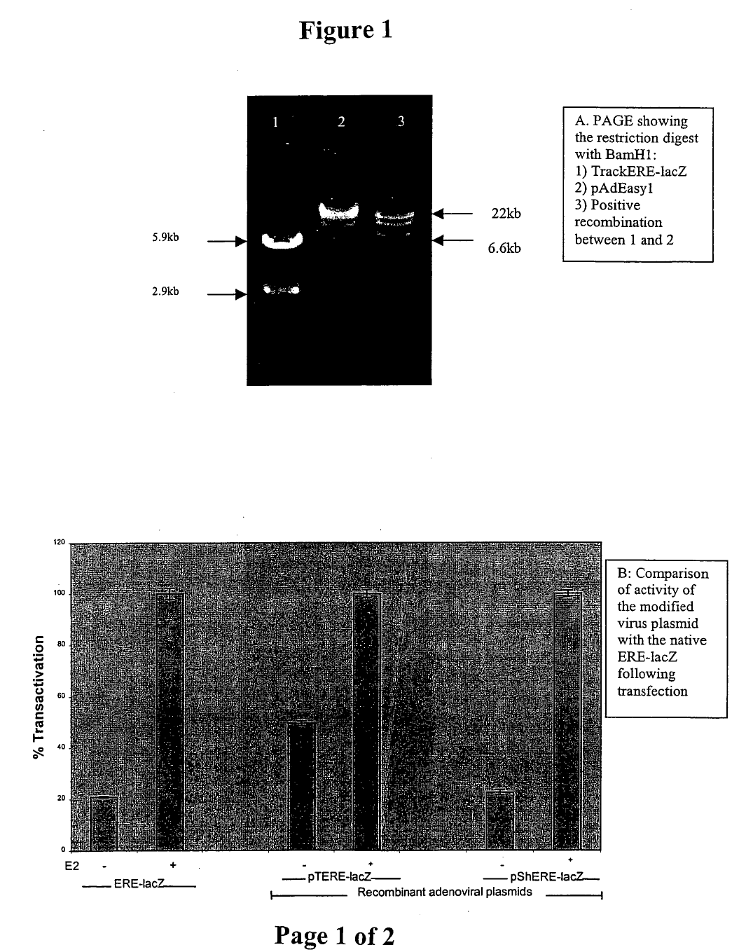

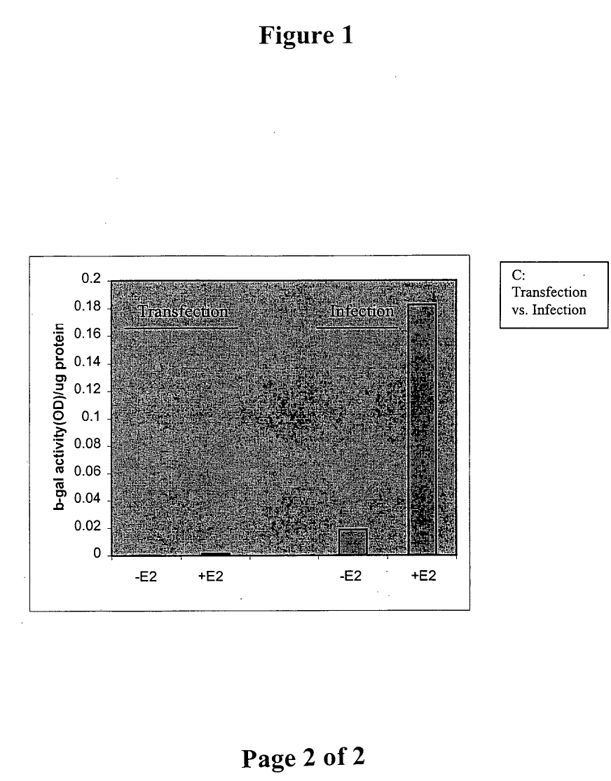

FIG. 1: A. PAGE showing the restriction digest with BamH1: 1) TrackERE-lacZ; 2) pAdEasyl; 3) Positive recombination between 1 and 2. B. Comparison of activity of the modified virus plasmid with the native ERE-lacZ following transfection. C. Comparison of Transfection and infection.

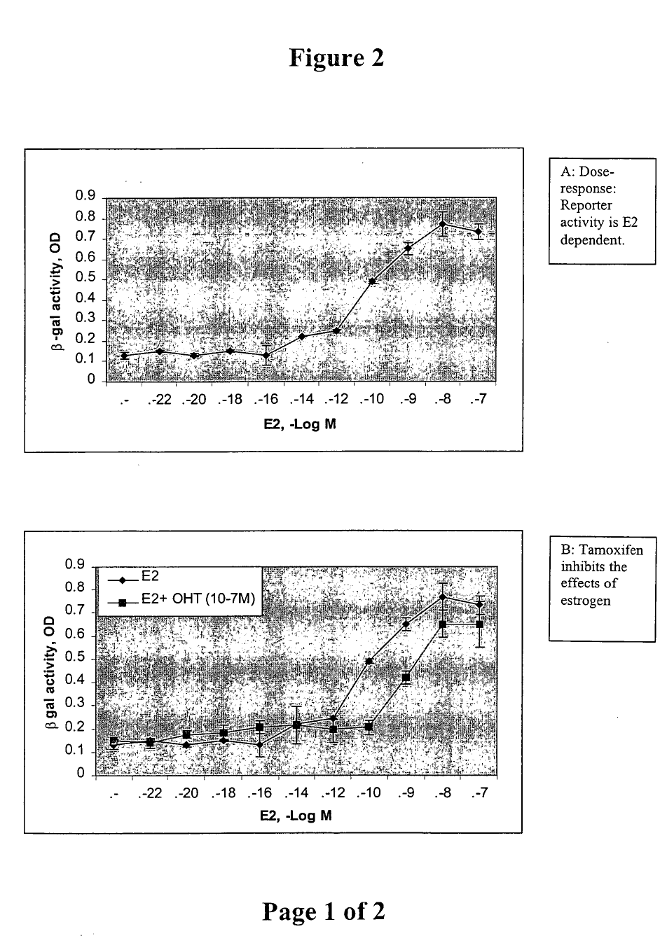

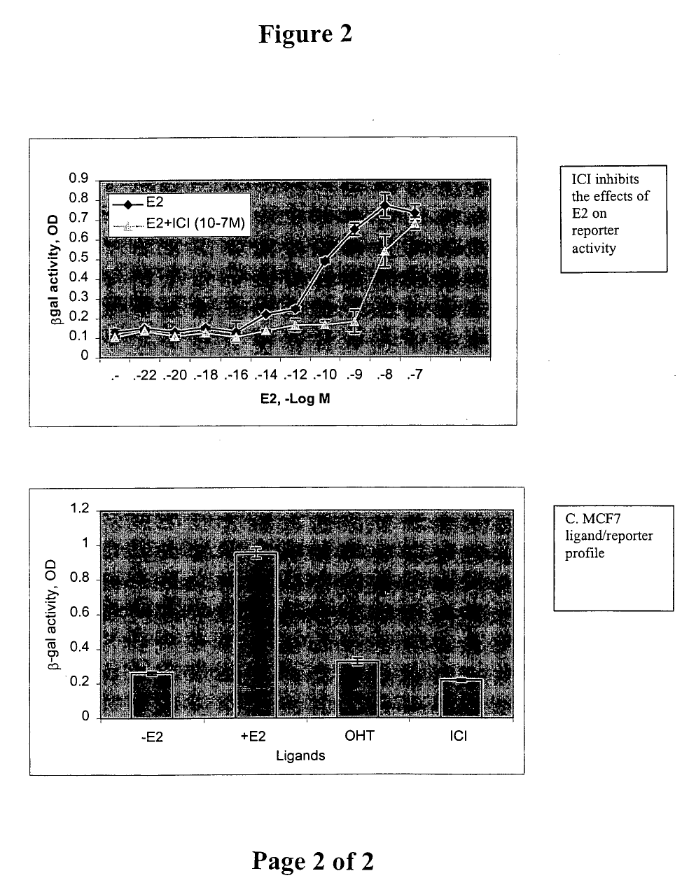

FIG. 2: A. Dose response: reporter activity is E2 dependent. B. Tamoxifen inhibits the effects of estrogen. C. ICI 182,780 (ICI) inbibits the effects of E2 on reporter activity. D. MCF7 ligand/reporter profile.

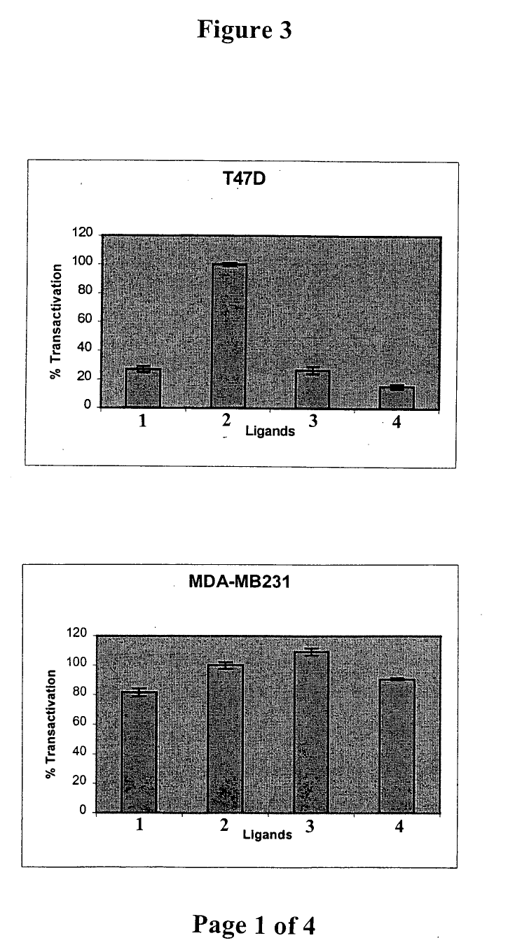

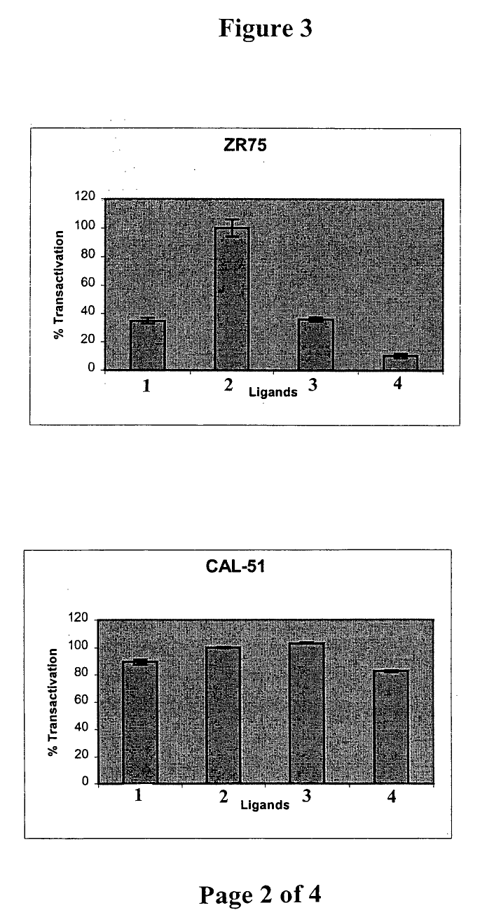

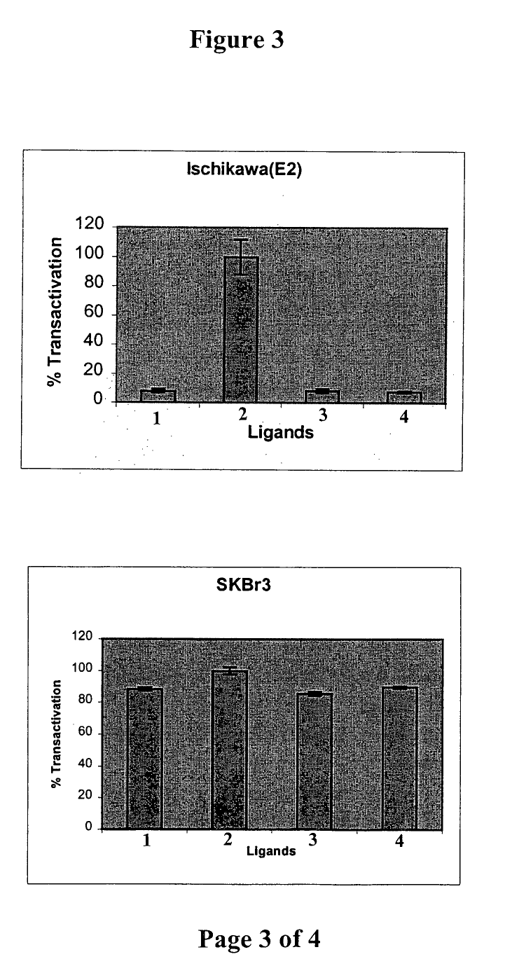

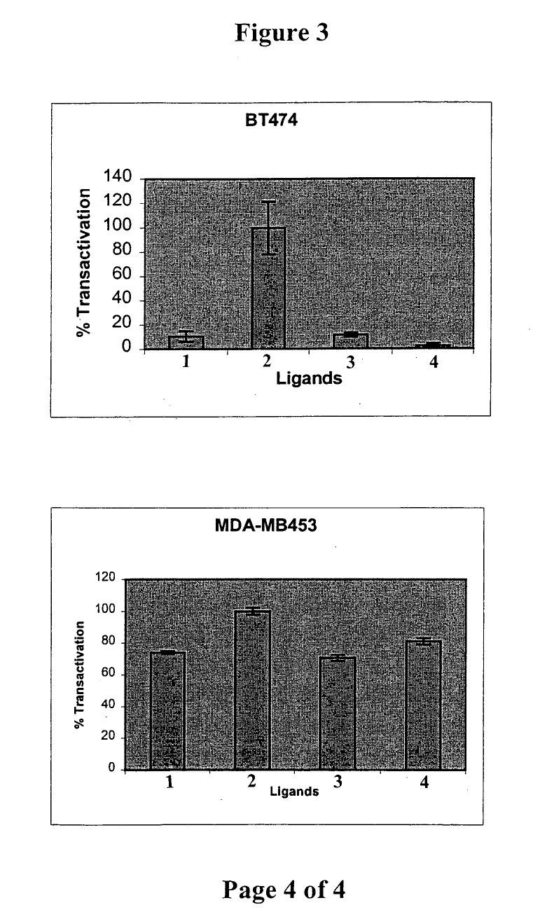

FIG. 3: Reporter activity in variety of estrogen receptor alpha positive (MCF-7, T47-D, ZR75-1, BT474) and negative (Cal-51, SkBr3, and MDA-MB231 cell lines). For each figure, the order is as follows: (1) refers to the no ligand control. (2)(B17B-oestradiol (3), 4-hydroxytamoxifen (4), and ICI182780 were added to a final concentration of 10⁻⁷M.

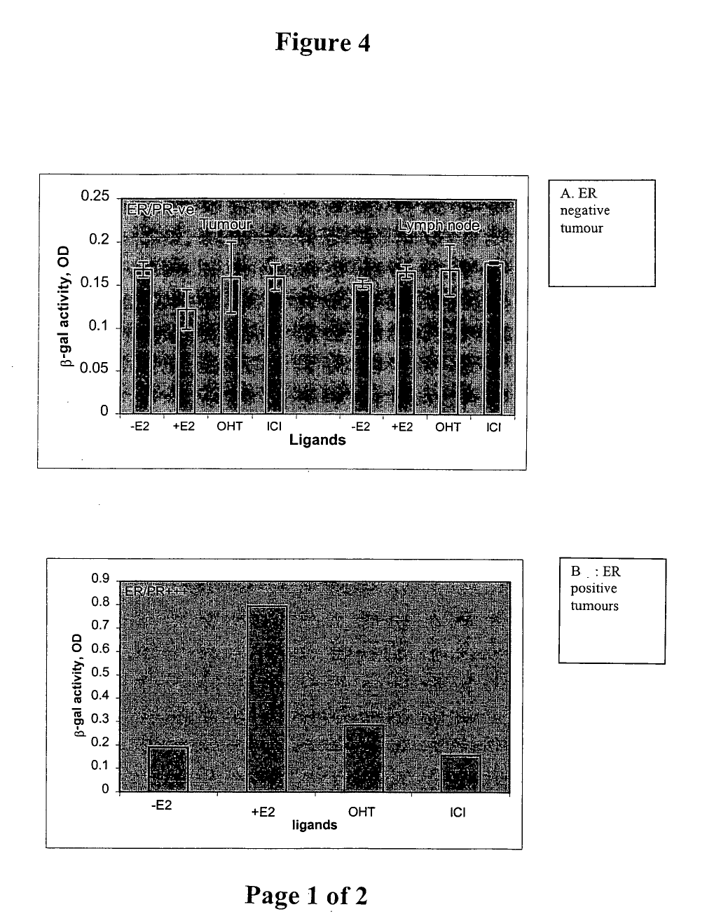

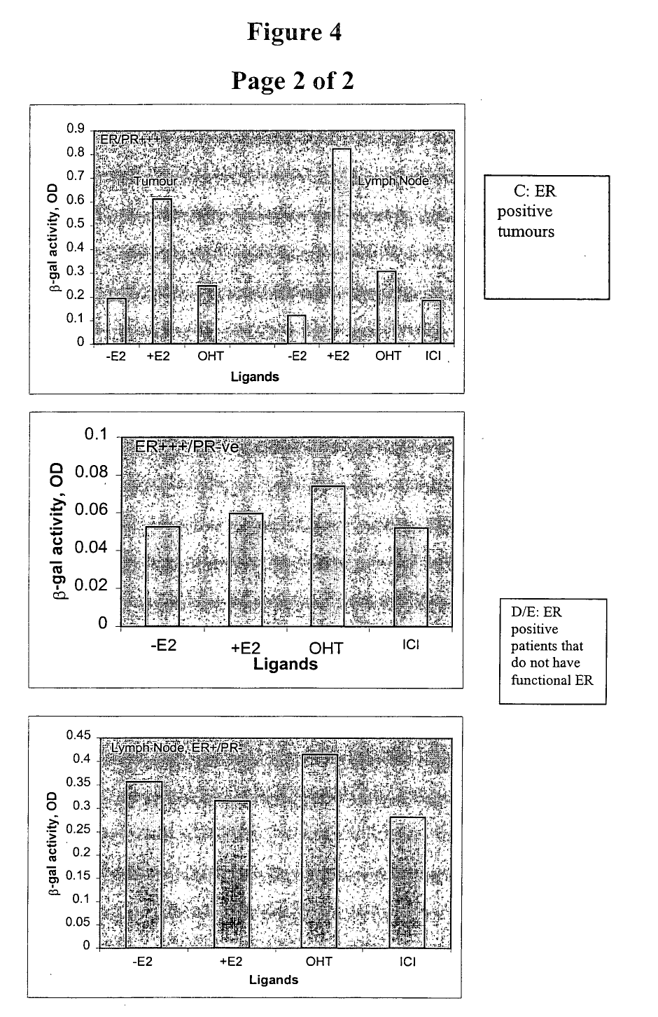

FIG. 4: Representative reporter activity profiles in purified primary breast cancer cells from a variety of patient samples. A. ER negative tumour. B/C. ER positive tumours. D/E. ER positive patients that do not have functional ER.

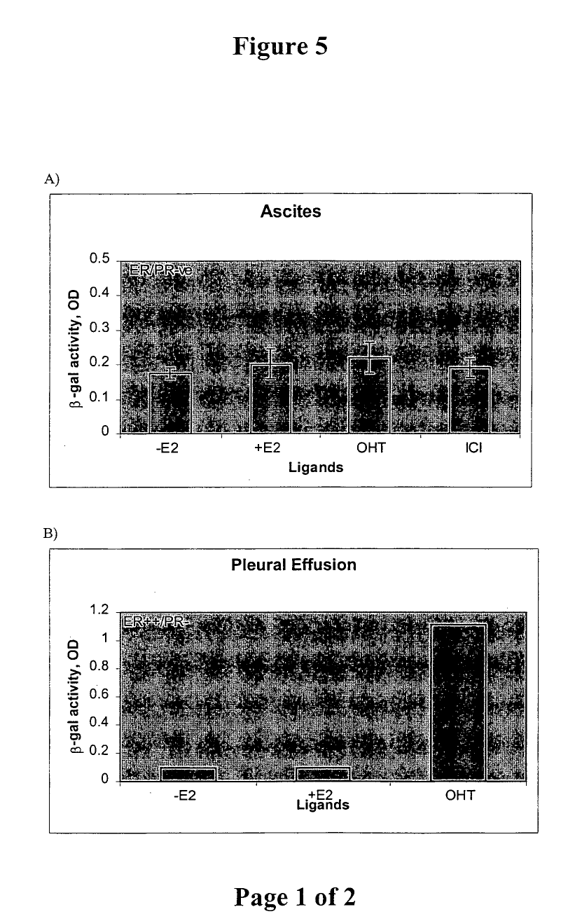

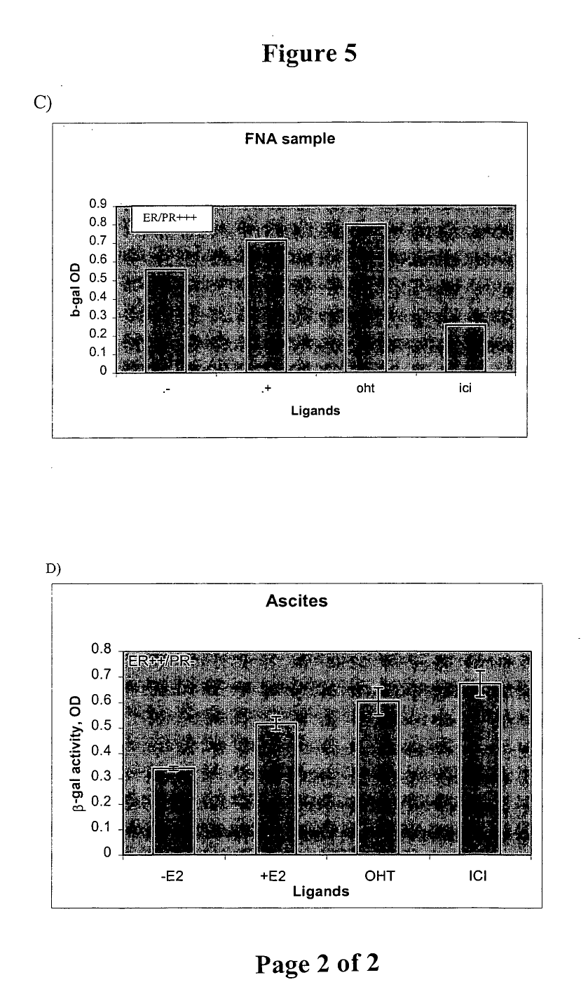

FIG. 5: Reporter activity in recurrent breast cancer. A Lymph nodes. B. Ascites. C. Pleural effusion. D. FNA sample. E. Ascites.

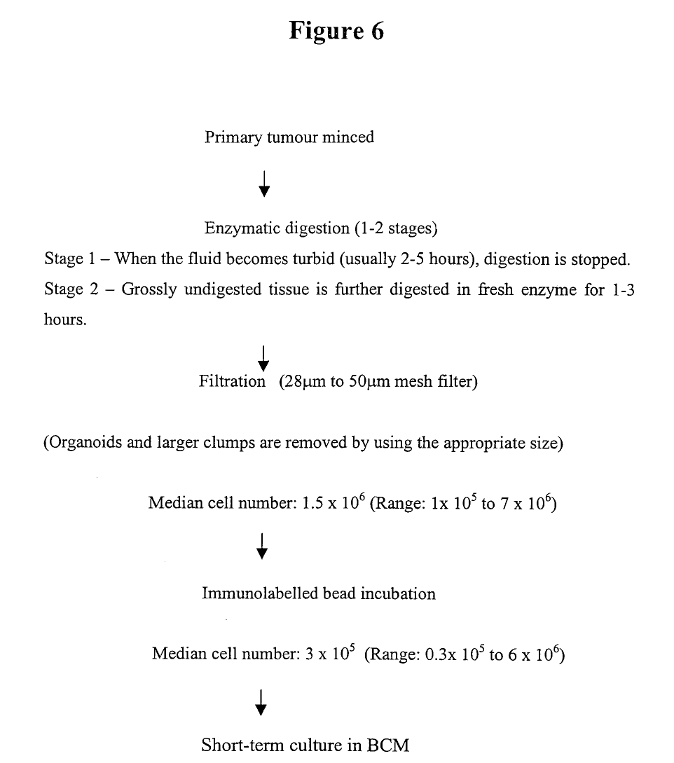

FIG. 6: Outline of the procedure for the isolation of pure populations of malignant cells from primary tumors. Each preparation is modified in terms of digestion time and the mesh sizes used in the filtration steps to suit the particular tumor.

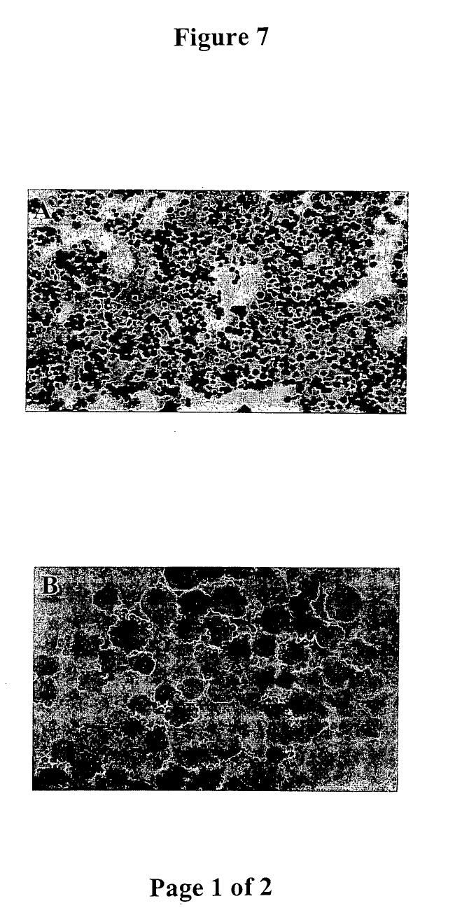

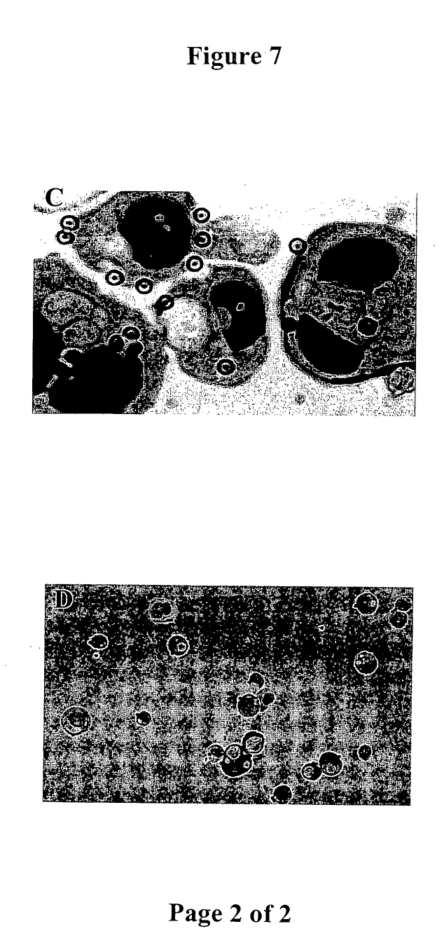

FIG. 7: Purified malignant epithelial cells from a primary tumor stained with May Grunwald Giemsa (MGG). A: Cell suspension prior to addition of immunolabelled Dynabeads (×100), B: Purified epithelial cells from (A) (×200), C: Purified epithelial cells from an intralobular cancer after 7 days in culture (×400) and D: Purified epithelial cells same as (C) after 7 days in culture (×200)

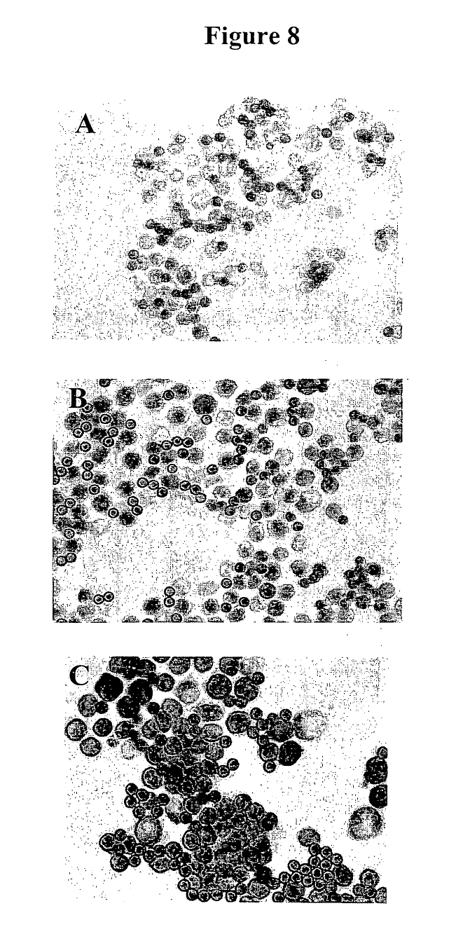

FIG. 8: Immunocytochemical staining of separated malignant epithelial cells from primary breast tumours. A ER staining, B, PR Staining and C pan-cytokeratin staining, (Magnification ×200).

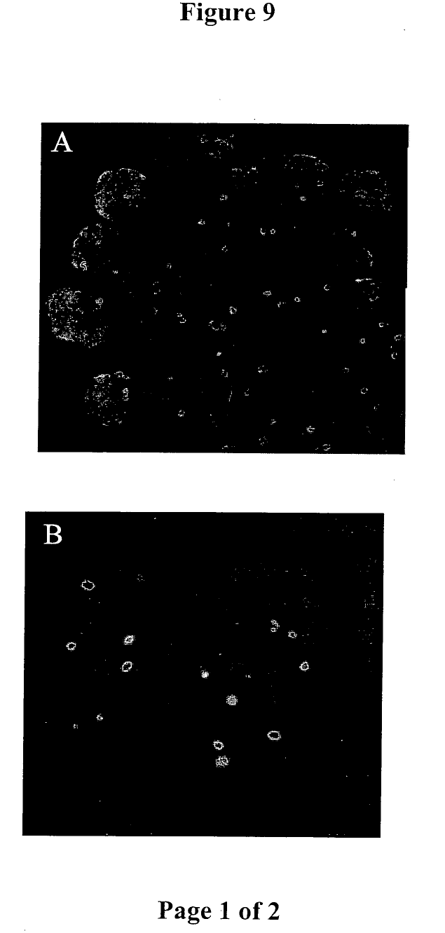

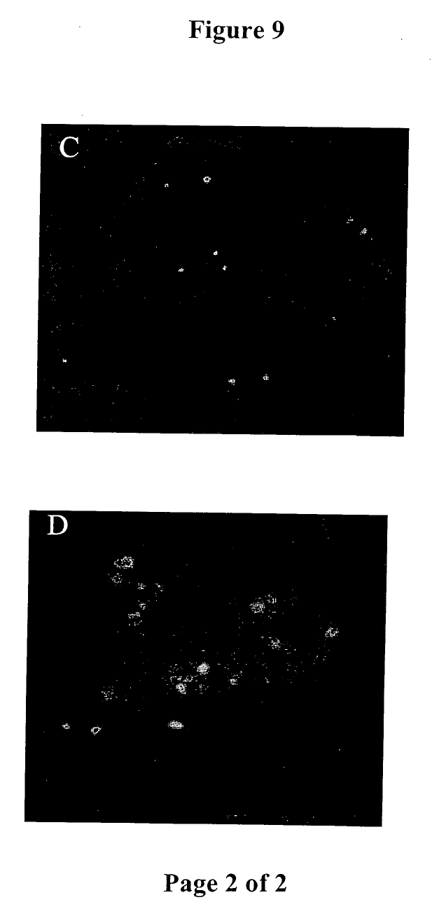

FIG. 9: Fluorescent in situ hybridisation of purified malignant epithelial cells from a primary tumor. A: Cell suspension prior to addition of immunolabelled Dynabeads (chromosomes 7 and 17), B: Purified epithelial cells (chromosomes 6 [red] and 11 [green]), C: Purified epithelial cells (chromosomes 7 [red] and 17 [green]) and D: Purified epithelial cells (chromosomes 12 [red] and 18 [green]).

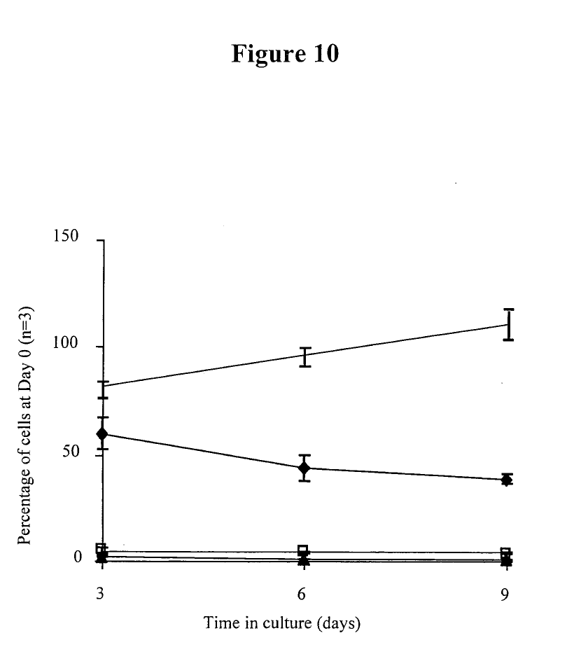

FIG. 10: Graph showing the viability of primary normal and malignant mammary epithelial cells over 9 days as determined by Trypan Blue staining and cell counting. The experiment started 3 days after the cells were seeded in order to allow for cell adherence.

The adherent normal epithelial cells (−) proliferate and hence the increase in numbers after the initial reduction between days 0-3. Approximately 60% of the malignant epithelial cells were adherent after 3 days and this fell to approximately 45% nine days after seeding (♦). Both the floating dead malignant cells (□) and the floating live malignant cells (▴) were less than 5% throughout the experiment.

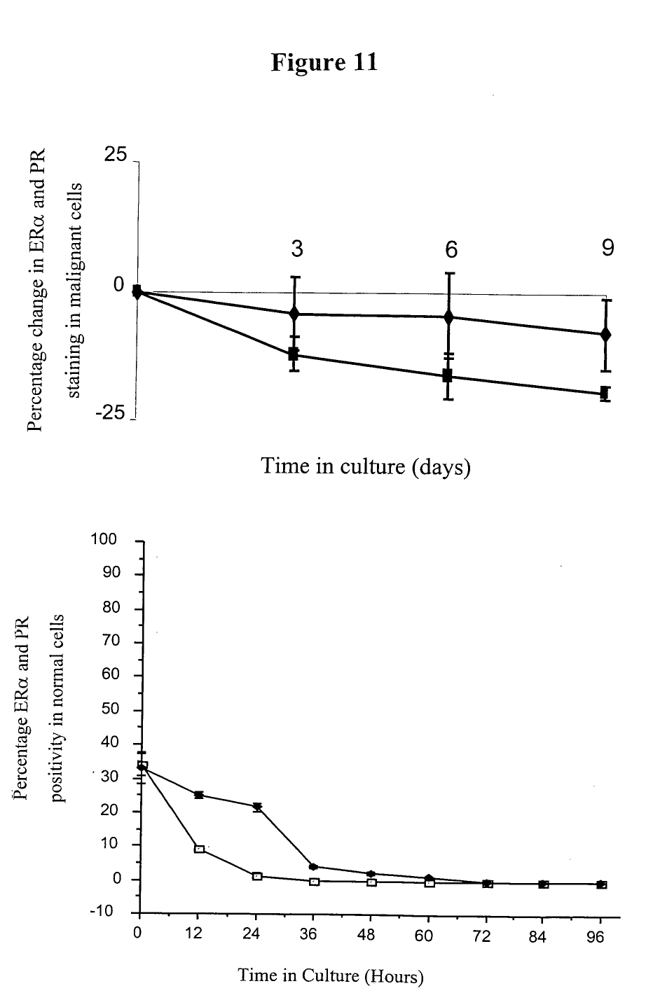

FIG. 11:

(a) ER and PR status of primary malignant mammary epithelial cells over 9 days expressed as the percentage change. There was a reduction in the percentage of cells positive for both ER and PR, however, the majority of the malignant cells remained ER and PR positive after 9 days in culture. (⋄) ER (n=6), (▪) PR (n=3) and (b) ER and PR status of primary normal mammary epithelial cells over 96 hours. Both receptors were rapidly lost in the normal cultures, so that after 36 hours in culture all the cells were ER and PR negative. (□) ER and (⋄) PR.

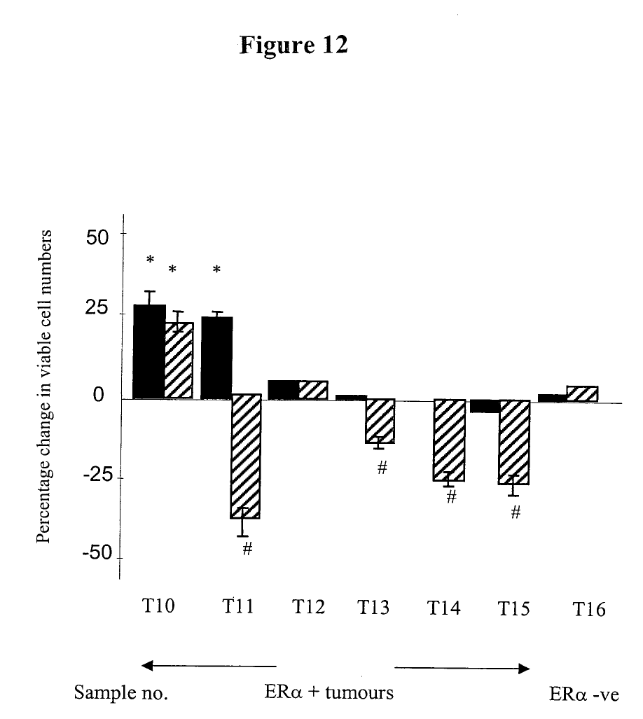

FIG. 12: The effects of estradiol and tamoxifen in terms of cell survival on malignant mammary epithelial cells (▪) Estradiol, ( [symbol] ) Tamoxifen, (*) Significant difference from ethanol effect, (#) Significant difference from estradiol and Tamoxifen effects.

### EXAMPLE 1

### A Reporter Gene Assay to Determine Estrogen Receptor Activity in Primary Breast Cancer Cells

Material and Methods

Cell Lines and Cell Culture

Human breast cancer cell lines MCF7, T47D, BT20 were maintained in DMEM (Sigma, Poole UK) supplemented with 10% fetal calf serum (FCS). ZR75, MDA-MB231, MDA-MB 435, SK-Br-3, CAL-51, HBL-100 were grown in RPMI-1640 (Sigma, Poole UK) containing 10% FCS. The human uterine cell line Ishikawa cells was cultured in DM-F12 containing 10% FCS. All cell lines were cultured at 37° C. in a humidified atmosphere with 5% CO₂. Ischikawa (−), IshikawaE2 cells were grown in DME-F12 (Sigma, Poole UK) medium supplemented with 10% FCS and in addition 10 nm E2 in the case of IshikawaE2.

Patient Samples

The study was conducted in accordance with the Declaration of Helsinki and was approved by the ethical review boards. All samples were obtained with consent. Breast tumours and lymph node samples were collected fresh from the pathologist. Metastatic effusions were collected from patients requiring drainage of effusions (pleural or peritoneal) for symptomatic relief. Fine needle biosies were performed with a 16G needle and 10 ml syringe using standard techniques.

Purification of Primary Breast Cancer Cells

The tumour was finely chopped using a disposable scalpel and digested with freshly prepared, sterile filtered collagenase (Sigma, Poole UK) made up in RPMI-1640/2% FCS at a concentration of 1 mg/ml, for 2-5 hours, at 37° C. in a shaker incubator. The product was then filtered through a nylon mesh (Lockertex Ltd, Merseyside, UK) and the filtrate centrifuged at 2000 rpm, at room temperature (RT). The pellet was re-suspended in 9 mls of RPMI/1% FCS and a cell count performed to estimate the number of tumour cells in the preparation. Magnetic beads coated with the Ber-Ep4 antibody (Latza, U et al. Ber-EP4: new monoclonal antibody which distinguishes epithelia from mesothelial, J. Clin Pathol. 43: 213-9, 1990. Dynal UK Ltd, Bromborough, Wirral, UK) were then added to the cell suspension at a ratio of 2:1 beads per tumour cell and incubated at 4° C. for 1 hour on a rolling shaker, after which, the suspension was applied to the magnet (Dynal) for 6 mins. The beads and attached tumour cells were washed 3 times on the magnet with RPMI-1640/2% FCS. A count of viable tumour cells was performed using trypan blue (Sigma, Poole, UK) prior to seeding in breast complete medium (DMEM/F-12 containing, 15 mM Hepes, 2 mM L-glutamine, 100U/ml penicillin, O. 1 mg/ml streptomycin, 50 U/ml polymixin B, 2.5 μg/ml amphotericin B, 5 μg/ml insulin, 10 μg/ml apo-transferrin, 100 μM ethanolamine, 1 μg/ml hydrocortisone, 10 ng/ml EGF) plus 5% FCS) for culture. The cells were used for experiments on day 3 after the initial plating.

In the case of metastatic fluid, 100 mls of the fluid was sent for cytological examination and staining for ER and PR. The remaining fluid was centrifuged and the cell pellets were pooled. This was re-suspended in RPMI-1640/1% FCS and tumour cell numbers estimated as before. If the cells were in clusters, trypsin/EDTA (Sigma, Poole, UK) was used to obtain single cell suspensions before filtering to remove any debris. The remaining steps were as above. The purified cells were either infected immediately or cultured for a few days prior to infection.

Cells obtained from FNAs were collected in PBS (Sigma, Poole, UK) containing 2% FCS. If the aspirate was bloody, erythrocyte lysis buffer (0.155M NH₄Cl, 10 mM KHCO₃, 0.1 mM EDTA, pH 7.4) was used to lyse the red blood cells and after a further wash with PBS, the cells were re-suspended in culture media for counting and immediate infections.

Adenovirus Production and Infections

Briefly, Sma1/Sal1 fragment containing 2 estrogen response elements linked to a β-gal reporter gene (ERE-lacZ) was sub-cloned into ECORV/Sal1 sites of the pShuttle and pAdTack vectors (He et al, 1998); ERE-lacZ was a gift from D. Rose (University of California, San Diego, Calif.). Recombinant viruses were produced in the BJ5183 bacteria. 100 ng of the replication deficient adenoviral vector pAdEasy-1 plasmid together with 100 ng of the linearised, modified pAdTrack or pShuttle vectors were transformed into the bacteria using the electroporation mehod. Positive recombinant clones were selected by their resistance to kanamycin. The recombinant DNA was extracted and used to transfect 293 human embryonic kidney cells (HK 293) cells, which express the E1A and E1B genes necessary for the start of virus propagation. Viruses were purified by ultracentrifugation using 2 caesium chloride gradients as described in He et al 1998. Viral titres were determined by plaque assays performed in HK 293 cells infected with the virus in serum free (SF) ×2 MEM medium (Gibco BRL), overlayed with agarose/2×MEM mixture and incubated for 7-10 days.

For adenovirus infections, cell lines were plated in 96 wells at the given density in phenol red free DMEM supplemented with 5% double-stripped serum (PR/5% DSS). The following day cells were exposed to the given quantity of plaque forming units per cell (multiplicity of infection, MOI) in PR/SF medium. This was incubated at 37° C. for 1 hour after which the media was replaced by PR free/5% DSS containing the ligands.

For infections of primary cells, 6-10,000 cells/well were allowed and exposed to the given quantity of virus, after which they were pelleted and re-supended in 200 ul of PR free/5% DSS medium containing the appropriate ligands prior to plating into 96-well plates. Ligands E2, 10 nM; OHT, 100 nM; ICI 182,780, 100 nM were prepared in ethanol.

Cells were incubated for 48 hours following infections unless stated otherwise.

Reporter Gene Assay 48 hours after virus infection, 20 ul of 20% Triton in PBS was added to each well and incubated at RT for 10 minutes. Chloro-phenol-red-guanothiocyanate (CRPG) made up in TBS at a concentration of 8 mg/ml was then added to the wells and the plate was incubated, foil-wrapped, at 37° C. until a colour change was observed. Plate readings were taken at a wavelength of 547 nm.

Results

Recombinant Adenovirus efficiently infects human breast cancer cell lines In order to study the feasability of determining ER activity in primary breast cancer cells, two recombinant adenovirus were constructed; the pShuttleERE-lacZ recombinant which contained 2 EREs located upstream of lacZ reporter and the pTrackERE-lacZ recombinant which bad in addition a CMV driven GFP gene located upstream from lacZ and ERE respectively for determination of adenovirus infectivity.

Successful recombinants were identified by their restriction digest patterns with several restriction enzymes. For example, with Pac1 a 3.0 kb fragment was produced in addition to the much larger pAdEAsy1 virus fragment and with BamH1, a 5.1 kb fragment containing the ERE-lacZ was produced in addition to the 11.7 and 21.7 kb fragments from AdEasy1 sequences (FIG. 1a).

The viral recombinants were tested for estrogen responsiveness prior to virus production. LacZ reporter activity in the presence of E2 was compared amongst ERE-lacZ, pTERE-lacZ and pShERE-lacZ plasmids. These plasmids, along with the GTK-CAT plasmid to control for transfection efficiency, were transfected into MCF7 cells using the calcium phosphate method and after 48 hours of incubation cell lysates were analysed for β-gal and CAT activity by ELISA kits. The results are shown in FIG. 1b. In the case of pShERE-lacZ recombinant, E2 induced a 5 fold increase in activity of b-gal relative to CAT and this is similar to that seen with the native ERE-lacZ. However, pTERE-lacZ showed a higher background b-gal activity with only a 2 fold induction with E2. Therefore, despite the attractive feature of constitutive GFP expression with this recombinant, it was not suitable for determining ER activity. Nevertheless, pTERE-lacZ was packaged for virus production and used in experiments to assess infection efficiencies. pShERE-lacZ was packaged and used to assess ER activity. FIG. 1c shows a comparison of the response of the reporter gene when delivered by transfection using fugene (most efficient transfection method in our hands) compared with adenovirus infection, in MCF7 breast cancer cells. B-gal activity in the presence of estrogen was 100 times more potent with adenovirus infection than transfection (greater absolute values and 10 fold increase rather than 5 fold with transfection). Indeed, using transfection we were unable to assess gene delivery in purified primary breast cancer cells (unpublished data), presumably due to poor efficiency of the transfection method (in the presence of relatively small number of cells). Table 1 shows a titration of the adenovirus in MCF7 cells. Infectivity was proportional to virus concentration and at a viral MOI of 250:1, more than 90% of the MCF7 cells were expressing GFP as determined by FACS analysis.

MCF7 Cells Infected with pShERE-lacZ Shows E2 Dependent Activation of the β-gal Reporter

To determine the conditions under which adenoviral infection is optimal in breast cancer cells, studies were performed to work out the appropriate concentration of adenovirus, the optimum ratio between cell numbers and virus MOI as well as the appropriate length of time between administration of virus and measurement of gene expression (data not shown). MCF7 cells were plated at 0.5×10⁵ cells per well in a 96 well plate, infected with a virus concentration of 100:1 and incubated for 48 hours prior to carrying out the reporter assay provided the optimum balance between infection efficiency and reporter activity. Under these conditions, 50% of the cells were infected. A β-gal assay based on CPRG was used for its sensitivity and simplicity. FIG. 2a shows that reporter activity was induced by E2 in a dose-dependent manner with a maximal activity at 10⁻⁹M E2. The competitive partial antagonist tamoxifen used at a concentration of 10⁻⁷M inhibited the effects of E2 up to a concentration of 10⁻⁹M in this system (FIG. 2b). This result is consistent with the binding affinities of estrogen and tamoxifen. Similarly the complete ER antagonist ICI 182, 780 (10⁻⁷M) also blocked E2 induced reporter activity.

The proportion of cells expressing β-gal following infection was demonstrated by X-gal staining of MCF7 cells. In the absence of E2, approximately 10% of the cells expressed mostly weak β-gal staining. However, E2 resulted in more intensely stained cells as well as increased number of cells. Tamoxifen induced a degree of β-gal activity but ICI completely abolished even basal levels of staining.

Thus the adenovirus system results in a highly efficient gene delivery whilst maintaining the response characteristics of the ER reporter in MCF7 cells.

Viral ER Reporter Reliably Predicts ER Activity in a Panel of Cell Lines

We went on to test the virus reporter in a variety of ER positive and ER negative cell lines. Again 0.5×10⁵ cells were infected with 100 virus per cell (100:1). Using the GFP virus it was evident that the infectability of the cell lines was variable. For instance whereas 50% of the MCF7 cells were infected at 100:1, >95% of the ER negative MDA-MB231 cells were infected and 75% of ER positive T47D cells were infected. To account for the variability in infection, β-gal activity is expressed relative to β-gal activity in the presence of E2 for each cell line. (It should be noted that varying the viral concentration for infection did not affect the response characteristic of the cell lines). FIG. 3 shows the ER reporter profiles of various breast cancer cell lines. In the ER positive cell lines, E2 produced 3.0 to 10.0 fold increase in reporter activity above baseline. 4-OHT had no significant effect above baseline whereas ICI blocked even the baseline activity.

Of the ER negative cell lines, MDA-MB231, MDA-MB435 and CAL-51 were in fact ERβ positive. The presence of E2 had no appreciable effect on the ERE driven β-gal reporter in any of the ER negative cell lines. As expected, in contrast to ER positive cell lines, ICI 182, 780 did not have a significant effect on the baseline activity.

Adenovirus Mediated Gene Delivery in Primary Breast Cancer Cells

We have developed a methodology to purify primary breast cancer cells from a variety of tissue samples including primary tumour, lymph nodes and metastatic effusions. A high degree of purity was reliably achieved using a combination of mechanical and enzymatic disaggregation followed by filtration and magnetic bead extraction of epithelial cells. Cytology and FISH analysis confirmed purity of above 95% for cancer cells obtained from 8 separate patient samples. The purified cells could be successfully maintained in short-term cultures for at least 10 days, which was more than sufficient time for the infection experiments. Importantly, using immunohistochemistry, we showed that ER and PR status was maintained over this period in all ER positive samples.

We aimed for a high purity of cancer cells in our preparations and this inevitably was at the expense of cell numbers. We therefore wished to determine the minimum numbers of cells which when infected would yield a reliable and reproducible reporter response. Once again these optimization experiments were performed in MCF7 cells. By titrating cell numbers with viral concentration, we estimated that around 6000 cells/well and a virus MOI of 1000:1 would be needed for determination of ER activity.

The number of cells obtained from each tumour sample varied from 0.24×10⁵-0.6×10⁶ and infectability was assessed in addition to reporter activity when cell numbers were sufficient to allow this. With purified primary breast cancer cells, as with cell lines, we found a variation in the infectability of breast cancer cells from different patients. However, despite this, we were reliably able to make an assessment of reporter activity using the CPRG assay. Replicates were performed when cell numbers would allow it.

We have used the adeno-reporter to infect purified cells from breast cancer and lymph nodes of 19 patients. The reporter assay was performed in a blinded fashion since the IHC report of ER status was not available until after the experiments were complete. Viral infections were performed in suspension so as not to loose cancer cells since they were only loosely adherent. Following exposure to the virus for an hour, the cells were washed to remove excess virus and then plated and incubated in PR free/5% DSS supplemented with the given ligands for 48 hours.

FIGS. 4a and 4b show typical examples of the reporter profile obtained for ER positive and ER negative patient samples. The absolute β-gal values are comparable to those seen with cell lines. Tamoxifen, a partial agonist was inhibitory in most cases but did show some agonistic activity in a proportion of patients. ICI was inhibitory in all cases. Overall there was agreement with IHC in 80% of the patient samples. In one of the patients, we were only able to assess cancer cells from the lymph node. This was found by our reporter assay to have an ER negative profile as seen in FIG. 4c. However, the tumour expressed both ER and PR. Re-examination of the lymph node for ER showed only weak staining for ER and no PR staining was observed. A second discordance with IHC was again in a PR negative but strongly ER positive patient. Despite reasonable infection efficiency (30%), the cancer cells expressed very little β-gal with a value of in the presence of E2. Thus although ER positive, these patients may not benefit from treatment with OHT. There was complete agreement with the PR status by IHC and our reporter assay.

ER Activity is Altered in Patients who have Relapsed Despite Prolonged Tamoxifen Therapy

Patients who have relapsed with breast cancer after previous tamoxifen treatment are an important group since their clinical response to tamoxifen is already known. We purified breast cancer cells from metastatic fluid, both pleural and ascites as well as fine needle aspirates (FNA) from recurrent breast cancer. These cells were counted and infected with adenoERreporter and after 48 hours a β-gal assay was performed. We have infected 9 metastatic effusions including 2 cell lines of passage number two, derived from pleural effusions and 4 FNAs. Tables 3 and 4 show the treatment history and characteristics of the patients that provided the samples. Where possible, some of the fluid was sent for cytology and ER staining. However, this was not always feasible due to the variability of cancer cells in the metastatic fluid.

FIG. 5 shows reporter activity in representative patient samples. The ER/PR negative cancers showed a profile, which was not different to the primary breast tumour (FIG. 5A). However, in the case of ER positive patients, E2 had less of a stimulatory effect on the reporter above the background activity, and the latter also tended to be higher (FIGS. 5B, C and D). Surprisingly, tamoxifen had an effect at least equivalent to E2 in patient samples 1, 5 and 6 (Table 3) and patient 6 (Table 4). In fact patient 1 showed approximately 10-fold increase in reporter activity in the presence of tamoxifen. ICI 182,780, a complete antagonist, which the patients had never been exposed to, was inhibitory in 50% of the cases.

Cells derived from patient 3, table 3, continued to respond to tamoxifen despite previous tamoxifen treatment. However, she received this for one year only when the average time of treatment is 5 years. Interestingly, patient 4 (table 3) who presented with ascites and had received no previous treatment showed a response profile similar to the cells derived from primary breast cancers. ER activity was related to the concentration of E2 in this patient, being maximal at a concentration of 10⁻¹⁰M E2. The latter corresponds closely to the serum level of E2 in post-menopausal women women (Coombes et al 1999. Endoc. Rel. Cancer 6/1259-63). OHT and ICI were both inhibitory to ER activity.

Discussion

This study is the first attempt to evaluate the transcriptional actuation of the estrogen receptor in patient biopsies. Previously, indirect methods were used to assess the function of the estrogen receptor. These methods include measurement of downstream genes such as progesterone receptor or PS2, as well as measures of ER binding to DNA (Liu et al, Cancer Res 2001, 61 5402-6). We have described an antiserum which recognises only the phosphorylated or ‘activated’ form or ER (Chen et al 2000 Mol Cell 6 (1), 127-137) but this and other methods are non-specific since both PR and PS2 induction and ER phosphorylation can be induced by factors other than activation of ER by estrogen. The most accurate method for assessing ER function hitherto has been to treat patients with breast cancer with an endocrine agent, either an antiestrogen or an aromatase inhibitor, and determine response by measuring shrinkage of primary of secondary tumours. However, this means delaying the administration of potentially effective chemotherapy, with the attendant risks of disease progression.

Our results indicate the following: firstly, we have shown that ER function is attenuated by endocrine treatment, since transcriptional activation the ER in biopsies from patients who have received endocrine therapy is clearly lower (at the ER concentration used in the assay) in biopsies demonstrating acquired resistance. Our failure to observe transcriptional activity equivalent to untreated breast cancers in recurrent tumours was not due to the presence of tamoxifen, which may have occupied the receptor thus preventing activation, since in the majority of cases tamoxifen had been discontinued many months before. Other explanations for lack of activity include the co-expression of dominant negative forms of ER such as ERβ CX (Ogawa et al. Molecular cloning. Nucleic Acids Res 1998. 26 (15): 3505-12); the absence, or mutation of, co-factors such as SRC-1 or other interacting factors such as components of TF11H (Chen et al 2000). Absent or reduced ER activity suggests that, in some cases, ER has a tumour suppressor effect, which may override other pathways leading to cell proliferation in tumours possessing a functional ER.

Irrespective of the cause of inactivity, ours is the first report to describe a reproducible method for assessing transcriptional activity in biopsy samples. The method is sufficiently sensitive to be done on small numbers of cells such as those obtained in fine needle aspirates and hence is capable of being used to ‘monitor’ the activity of the ER throughout the course of the disease. Up until now, it has been assumed that the ER, since its expression is retained, remains transcriptionally active and for this reason, pharmaceutical companies and other scientific groups have focussed on strategies to develop more therapies targeted at the estrogen receptor. Our results indicate that this is unlikely to be successful and a more effective strategy will be to target other mediators of growth.

Another significant finding is that ER in a subpopulation of primary, untreated breast cancers is inactive. Thus, it will be important to determine whether this correlates with failure to respond to primary endocrine therapy; nearly 50% of patients with ER positive tumours fail to respond to tamoxifen (Gazet et al 1987) and 40% fail to respond to aromatase inhibitors (Ellis et al 2001). This test could provide better discrimination for patients who would benefit from such therapy.

One unexpected finding was the observation that tamoxifen appears to be equivalent to estrogen in activating the estrogen receptor in some patients. This was in marked contrast to the tumours that were ER negative, where we consistently observed complete lack of ER transcriptional activity. This implies that, in many cases, tamoxifen is capable of driving ER mediated action when its proliferation effects are no longer present. In many of these tumours the pure antiestrogen ICI 182780 was able to abolish estrogen actions, suggesting that the test could be used as an invitro system to select patients for ICI 182780 (Faslodex) therapy after failure of tamoxifen. This finding also implies that, in some patients who are receiving tamoxifen as adjuvant therapy after primary breast cancer treatment, in an attempt to prevent the development of overt metastatic disease, tamoxifen could be acting as a mitogen during part of the 5 year course of treatment that is now given to all patients with ER positive breast cancer. This finding highlights the importance of finding a method of monitoring micrometastatic disease and also suggests that an aromatase inhibitor (and/or LH2H agonist in premenopausal patients) may be preferable.

It has been postulated that ERβ may be responsible for endocrine resistance but the role of ER beta is unclear since many of the patients with negative transcriptional activity actually possess tumours which contain ERβ. It may be that ERβ is inactive because of the co-expression of dominant negative ERβ receptors; alternatively, since we and others (Pace et al, 1998) have shown that ERβ and ERα heterodimerise, it may be that the presence of functional ERα is necessary for ER activation of transcription.

What is the possible clinical consequences of this functional assay? Until now, we have no way of determining which patients should have primary endocrine therapy and which patients, when they develop metastatic disease after relapse, should have endocrine therapy rather than chemotherapy. Using this assay, which can be done on as few as 6,000 cells (or less), easily obtainable in a fine-needle-aspirate, we have a more accurate predictive test. A prospective study is useful in which the test is done prospectively on ER patients and subsequently patients are be treated with endocrine agents and the outcome completed with the result of the Functional Estrogen Receptor Assay.

TABLE 1
Infection of MCF7 cells with AdGFP

| Adenovirus type | Virus MOI | % Fluorescing cells |
|---|---|---|
| Control virus (ad∅) | 500:1 | 2.22 ± 1.94 |
| adenoGFP | 10:1 | 12.9 ± .21 |
| | 25:1 | 21.1 ± 1.0 |
| | 50:1 | 41.3 ± 3.6 |
| | 100:1 | 75.4 ± 4.1 |
| | 250:1 | 94.3 ± 3.7 |
| | 500:1 | 93.0 ± 3.2 |

MCF7 cells plated at a density of 0.5×10⁶ cells/well were exposed to the indicated virus for 2 h after which they were incubated for 48 hours in growth medium prior to harvesting and FACS analysis for assessment of infectivity. Data represent mean±s.d. of at least 3 independent experiments.

TABLE 2

| | | Steroid | | | | | |
|---|---|---|---|---|---|---|---|
| | | Receptor | | | | | |
| | | status (by | Menopause | Infectivity | Reporter response | | |
| Patient | Histology | IHC) | status | of cells (%) | E2 | 4-OHT | ICI172,780 |
| 1 | | ER+++/PR+++ | Post | 10 | x1.5↑ | — | — |
| 2 | | ER+/PR++ | ?(49y) | 20 | x3.6↑ | inh | — |
| 3 Tu | | ER++/PR++ | ?51y | — | x1.4↑ | inh | — |
| Ln | | — | | — | x5.3↑ | x1.1↑ | inh |
| 4 Tu | | ER+++/PR+++ | Post | — | x3.1↑ | inh | — |
| Ln | | — | | 30 | x6.8↑ | x2.5↑ | x1.5↑ |
| 5 Tu | | ER+++/PR+ | | — | — | — | — |
| Ln | | ER+/PR− | Pre | 20 | ns | ns | ns |
| 6 | | ER+++/PR+++ | ? | — | x2.2 | inh | inh |
| 7 | | ER+++/PR+++ | Post | 20 | x4.2↑ | x1.5↑ | inh |
| 8 | | ER+++/PR− | Post | 30 | ns | ns | ns |
| 9 | | ER+++/PR+ | ? | — | x3.5↑ | inh | — |
| 10 | | ER+++/PR+ | Post | | x5.1↑ | x1.7↑ | — |
| 11 | | ? | ? | | x6↑ | x1.3↑ | inh |
| 12 Tu | Anaplastic | ER−/PR− | ? | — | ns | ns | ns |
| Ln | small cell | — | | 25 | ns | ns | ns |
| | carcinoma | | | | | | |
| 13 | | ER−/PR− | Post | — | ns | ns | ns |
| 14 | | ER−/PR− | Post | — | x1.3↑ | x1.2↑ | |
| 15 Tu | | ER−/PR− | ? | — | ns | ns | ns |
| Ln | | — | | 15 | ns | ns | ns |
| 16 Tu | | ? | Post | — | ns | ns | ns |
| Ln | | — | | | ns | ns | ns |
| 17 | | ? | ? | — | ns | ns | ns |
| 18 | | ER−/PR− | ? | — | ns | ns | ns |

TABLE 3

| | | | | Endocrine | | | |
|---|---|---|---|---|---|---|---|
| | Source | Receptor status | Receptor status | Treatment | | | |
| Patient/ | of | of primary | of recurrent | History | Reporter Activity | | |
| Histology | Sample | cancer | cancer | Prior to assay | E2 | 4-OHT | ICI |
| 1) T2G2NxM0 | PE | ER++/PR− | N/a | Tamoxifen 3y | Ns | x5↑ | x1.7↓ |
| | | | | Lentaron 5m | | | |
| 3) TG2N1M? | PE | ER+++/PR− | ER+++/PR− | Tamoxifen 1y | x2↑ | x1.34↑ | x0.65↓ |
| | | | | Lentaron 5m | | | |
| | | | | Arimidex 1y | | | |
| 5) T3GNxMo | PE | ER+++/PR+++ | N/a | Tamoxifen 5y | x1.2↑ | x1.6↑ | x1.6↓ |
| | | | | Arimidex 1y | | | |
| 4) M1 | Ascites | ER+++/PR+++ | N/a | Nil | x10↑ | x1.2↑ | x1.6↓ |
| 6) T4G2N1M0 | Ascites | ER+++/PR+ | N/a | Tamoxifen 2y | x1.5↑ | x1.8↑ | x2.0↑ |
| | | | | Lentaron 1m - NR | | | |
| | | | | CXT | | | |
| 7) T4G3N1M0 | Ascites | ER−/PR− | ER−/PR− | Nil | x1.2↑ | x1.1↑ | x1.0 |
| 8) T1G1N1M0 | Ascites | ER−/PR+ | ER++/PR− | LHRH 11m | x2.3↑ | x2.1↓ | ↓↓ |
| | | | | Arimidex - ?time | | | |
| 8) T1G3N1M0 | PE | ER++/PR− | ER+/PR− | Tamoxifen 3.5y | x1.26↑ | x1.1↓ | x1.2↓ |
| | | | | Lentaron 8m | | | |
| | | | | Arimidex 3m | | | |
| | | | | CXT | | | |
| 9) T2G3N1M0 | PE | ER−/PR− | ER−/PR− | Tamoxifen 2y | x1.2↓ | x1.1↓ | x1.2↓ |
| | | | | CXT | | | |

TABLE 4

| | | | Endocrine | | | |
|---|---|---|---|---|---|---|
| | Receptor status | | treatment history | | | |
| Patient/ | of primary | Receptor status | prior to Reporter | Reporter Activity | | |
| Histology | cancer | of FNA | assay | E2 | 4-OHT | ICI |
| 1) | ER−/PR− | ER−/PR− | Nil | Ns | x1.8↓ | x1.8↓ |
| T3G2N1M0 | | | | | | |
| 2) | ER+++/PR− | ER+++/PR− | Arimidex 1.5y | x1.3↑ | x1.3↓ | — |
| T2G2N1M0 | | | Megace - stable | | | |
| | | | ?progressing | | | |
| 3) | ER+++/PR− | ER+++/PR− | Tamoxifen 7m | x1.1↑ | x1.2↓ | x1.5↓ |
| T4G2N2M0 | | | Arimidex 7m | | | |
| (No surgery) | | | Megesterol - | | | |
| | | | stable | | | |
| 4) | ER−/PR− | ER−/PR− | | x1.1↑ | x1.3↓ | — |
| 5) | ER+++/PR− | | Nil | x1.2↑ | x1.2↑ | x1.1↑ |
| T5G2N1M0 | | | Rnd to FEC | | | |
| 6) | ER+++/PR++ | ER+++/PR− | Tamoxifen 5y | x1.3↑ | x1.4↑ | x2.2↓ |
| T2G3NxM0 | | | Megace 4y | | | |
| | | | Sx/DXT/Tam 2y | | | |
| | | | Tam cont. + | | | |
| | | | Arimidex 1y | | | |
| | | | Megace | | | |
| 7)T4N1GM1 | ER−/PR− | | Nil | x1.2↓ | x1.1↓ | x1.2↑ |
| 8)T2GN0M0 | ER++/PR− | ER−/PR− | Arimidex 1y - | x1.4↑ | x1.1↑ | x1.0 |
| | | | stable | | | |
| | | | For surgery | | | |
| 9) | ER+++/PR+ | | | x2.5↑ | x1.1↑ | x1.6↑ |

| Primary Tumours | IHC | Reporter Assay |
|---|---|---|
| N = 9(8) | ER+/PR+ | All responded |
| N = 2 | ER+/PR− | No response |
| N = 5(7) | ER−/PR− | No response |

| Metastatic effusions | IHC | Reporter Assay |
|---|---|---|
| N = 3 | ER+/PR+ (ascites) | Responders |
| | ER+/PR− (PE) | |
| | ER+/PR− (Ascites) | |
| N = 3 | ER+/PR− (PE) | Resistant |
| | ER+/PR− (PE) | |
| | ER+/PR+ (ascites) | |
| N = 3 | ER−/PR− | Non-responders |
| | ER−/PR− | |
| | ER++/PR− | |
| N = 3 | ER+/PR+ (ascites) | Responders |
| | ER+/PR− (PE) | |
| | ER+/PR− (Ascites) | |
| N = 3 | ER+/PR− (PE) | Resistant |
| | ER+/PR− (PE) | |
| | ER+/PR+ (ascites) | |
| N = 3 | ER−/PR− | Non-responders |
| | ER−/PR− | |
| | ER++/PR− | |

| FNAs | IHC | Reporter Assay |
|---|---|---|
| N = 2 | ER−/PR− | Non-reponders |
| | ER+/PR− | |
| | ER+/PR− | |
| | ER+/PR− | |
| N = 1 | ER−/PR− | ?Responder: |
| | | OHT/ICI inhibit by × 1.8 |
| N = 1 | ER+/PR+ | Responder |
| N = 1 | ER+/PR+ | Resistant |

### EXAMPLE 2

### Further Characterisation of Patients

A group of 60 patients with large (<4 cm) or locally advanced breast cancer is characterised for their response to primary tamoxifen therapy and this is related to the functional Estrogen Receptor Assay (FERA) as assessed by the assay performed on fine-needle-aspirates. All patients will commence endocrine therapy with Tamoxifen for three months followed by definitive surgery. Fine needle biopsies will be performed with a 16G needle and 10 ml syringe using standard techniques. Cells obtained from FNAs are collected in PBS containing 2% FCS. Further details of the FERA are described in Example 1.

### EXAMPLE 3

### Purification of Breast Cancer Cells

We show, in this example, that enzymatic treatment of breast cancer, followed by immunomagnetic bead cell purification, yields extremely pure populations of breast cancer cells, as judged by cytology and fluorescent in situ hybridisation (FISH). These methods have been developed as a direct extension of our studies of normal breast cell purification (Gomm et al 1995; 1997) and demonstrate the value of immunomagnetic bead purification for both normal and neoplastic breast cells. Other results presented here demonstrate distinct differences in retention of ER expression in normal and cancer cells with normal cells losing ER expression within hours of purification. However, despite loss of ER immunostaining in normal cells, we have evidence of retained ER expression since the normal cells respond to estrogen exposure by expressing both PR and PS2, both of which are known to be estrogen-regulated. Further, the cells show an increase in proliferation in response to estrogen and a decrease following Tamoxifen administration. In contrast, ER positive purified breast cancer cells show a reduction in cell death following estradiol reversed by Tamoxifen, but in no case did we observe proliferation of breast cancer cells. It is of interest that no effect of estrogen was seen in the ER negative breast cancer cells.

With regard to the observation that ER expression is rapidly attenuated in normal cells, several mechanisms may be relevant. Firstly, growth factor signalling is known to regulate ER expression through unclear mechanisms; the transcription factor AP2 [symbol] is known to be important and the levels and activation status of AP2 [symbol] could differ between normal and cancer cells. Many recent studies have suggested a link between transcription factor activity and their rate of degradation, with greater activity equating with deceased protein stability. ERot is degraded via the 26S Proteasome pathway and ubiquitination is influenced by ligands, unliganded receptor having a short half life. It is hence possible that breast cancer cells, by virtue of their elevated aromatase content, have a higher likelihood of possessing ligand-occupied ER (Wijayaratne and McDonnel, 2001).

Drawbacks of the immunomagnetic technique are the small numbers of cells obtained and also the laborious and time-consuming nature of the procedure. However, we believe the magnetic bead step is an essential component of our studies since the purification ensures that neither fibroblasts nor myoepithelial cells from the non-malignant elements outgrows the cultures: the comparatively low yield of cells has, however, necessitated the development of methods for studying small numbers of cells, in particular viral infection, still more particularly adenoviral infection (see Example 1). Unlike other groups (eg Dairkee et al, 1997) we have never observed proliferation of the purified neoplastic cells, in contrast to the normal luminal cells, which increased in number in the culture conditions used (Gomm et at, 1997). This considerably limits the scope of experiments that are possible; in particular, it precludes the testing of antiproliferative agents. The marked reduction in cell number also raises the possibility that we are studying a selected subpopulation of cells. We think this is unlikely, however, since both the FISH analysis and estrogen receptor status reflected the original primary tumour characteristics.

Other groups have published methods for the purification of breast cancer cells. These include Speirs et al (1998) who used differential centrifugation (Emerson and Wilkinson, 1990) to purify three populations of cells-organoids, epithelial and stromal. They used PCR for a calmodulin-related gene, NB-1, to check for contamination by normal, but not malignant breast cells (Yaswen et al 1990; 1992; Stampfer and Yaswen, 1993). However, using this technique, only approximately 50% of cells expressed epithelial membrane antigen (EMA). Overgrowth by non-epithelial cells is also suggested by their observation that ER expression was gradually lost over time. In contrast to the method of Dairkee et al (1997), where contamination by fibroblasts was a problem, necessitating differential trypsinisation, Speirs et al did not use trypsinisation to eradicate fibroblasts.

To overcome some of these problems, we devised a method an immunoaffinity purification procedure using magnetic beads then resulting in a very pure population of epithelial cells, as judged by PCR for luminal epithelial and myoepithelial markers (EMA and CALLA). Our recent studies have demonstrated that these two populations of cells have distinct growth medium requirements in that epithelial cells require foetal calf serum, EGF and FGF whereas myoepithelial cells are growth inhibited by foetal calf serum. (Gomm et al, 1997). We took these findings into consideration when choosing media for our cells.

Given the limitations in cell numbers obtained, we have established several characteristics of these cells, including viability, cytogenetics, ER/PR status and response to estrogen. In Example 1, we have also examined the ability of the cells to transcribe the β-galactosidase gene, introduced in an estrogen-inducible construct via an adenoviral vector. The results obtained indicate that there is a good correlation between these results and those obtained in Example 1.

One major problem besetting oncologists at the present time is to distinguish patients whose tumours contain functional, as opposed to non-functional ER, since in the latter population tamoxifen or aromatase inhibitors would not be expected to inhibit proliferation of any residual cells after primary surgery. This in vitro test may help distinguish these two populations; a patient whose cells did not respond to estradiol by a reduction in cell death would not be expected to benefit from adjuvant endocrine therapy, and would more likely require cytotoxic chemotherapy. This hypothesis may be validated using much larger numbers of patients in a prospective randomised trial designed to examine whether this test is capable of selecting patients more accurately for potentially curative adjuvant therapy. However, given the morbidity associated with cytotoxic chemotherapy, and the tendency to give both cytotoxic and endocrine therapy to increase numbers of patients, this test may well prove of value in a subset of patients with ER positive tumours in that it may prevent the need for chemotherapy in these patients.

Introduction

The human mammary gland is comprised of two epithelial cell components, namely luminal epithelial and myoepithelial cells, both of which can be purified by immunoaffinity techniques which exploit differences in marker cell surface protein expression (Clarke et al., (1994) Epithelial Cell Biol. 3: 38-46; Gomm et al. (1995) Anal Biochem. 226: 91-99; Gomm et al. (1997) J. Cell Physiol. 171: 11-9; Kamalatiet al. (1999) J. Mammary Gland Biol Neoplasia. 4: 69-77; Niranjan et al. (1995) Development. 121: 2897-908, 1995; Page et al. (1999) Proc Natl Acad Sci USA. 96: 12589-94; Slade et al. (1999) Exp Cell Res. 247: 267-78). Using such protocols it has been possible to further study and highlight the responses of these two cell types to growth and morphogenic signals, thereby gaining further insight into the functionality of these cells in normal mammary gland function. Furthermore, attempts to obtain cell lines from primary normal human mammary epithelial cells that retain normal characteristics have largely been unsuccessful, highlighting the need for further development of the purification protocols so as to obtain robust and easily used methodologies.

It is thought that luminal epithelial cells are the main precursor cell population from which breast cancers arise, although gene expression studies indicate that there may be myoepithelial gene expression signature in a significant number of such tumours (Perou et al. (1999) PNAS. 96: 9212-9217). Nevertheless, comparison of tumour and normal highlight estrogen receptor α expression in breast cancer, a protein found to be expressed only in the luminal epithelial component of the normal mammary gland. Estrogen receptor α (ERα) is expressed in 50-70% of all breast cancers, where endocrine therapy directed at inhibiting ERα function, is used as a treatment following surgery. From this, and through observations in animal models it is clear that estrogen is an important growth and/or survival factor in breast cancer. However, the response of primary human breast tumour cells to estrogens and anti-estrogens remains poorly studied, primarily due the paucity of ERα-positive breast cancer cell lines and the lack of methods for obtaining primary human breast tumour cells. These problems have prompted us to evaluate the potential for immunoaffinity purification to characterise primary human breast tumour cells. In doing this we sought to modify our original protocols for the purification normal mammary epithelial cells so that they can be applied to both the normal and malignant mammary gland. By exploiting the expression of epithelial cell adhesion molecule antigen, found on the surface of primary luminal epithelial and tumour cells we have been able to obtain near homogeneity purifications for both cell types. Surprisingly, we find that even though both tumour and normal cells are purified using the same protocol, ERα expression in primary normal cells is unstable and is rapidly deattenuated. By contrast ERα expression is stable and can be maintained in cultures of purified malignant epithelial cells. Finally we show that, under conditions which stimulate growth of the normal cells purified, tumour cells do not proliferate and remain in this quiescent state, even following treatment with estrogen.

Materials and Methods

Fresh tissue was obtained from 31 cases of primary breast carcinoma (Table 5) and 6 cases of reduction mammoplasty, which on subsequent histopathological examination showed no signs of malignancy or other abnormality. All patients were treated at Charing Cross and West Middlesex Hospitals, London and gave written, informed consent as required by the local Ethics Review Board.

Purification of Malignant Epithelial Cells from Cancers

Sterility was maintained at all times, throughout tissue handling and transportation.

The tumour tissue was minced and digested in centrifilge tubes with type IA collagenase (1 mg/ml) (Sigma Chemicals) in RPMI-1640 media supplemented with 5% Foetal Calf Serum, penicillin, streptomycin and amphoterecin B in a shaking incubator at 37° C. When the solution became turbid (usually 2-5 hours), digestion was stopped temporarily and loose cells and clumps including some organoids filtered out using a 50 μm pore nylon mesh (Lockertex). Tissue remaining on the mesh was redigested once for 1-3 hours and filtered through a 50 μm pore nylon mesh. The filtrates were then pooled and filtered again using a range of nylon mesh filters between sizes 50 μm and 28 μm to remove larger clumps and some ‘organoids’ that would easily pass through a 50 μm mesh. This is an essential step that helps remove most normal epithelial cells that are in the form of organoids or clumps and would have overgrown the malignant cells in culture. The filtrate now contains predominantly single cells including malignant cells, macrophages, lymphocytes, red cells and some small clumps of up to 5-10 cells whereas the normal epithelial cells, which are in the form of ‘organoids’ at this stage of digestion, are retained on the mesh.

Cell suspensions from the filtrate were then incubated with commercially available superparamagnetic, polystyrene beads (Dynal, Wirral, UK) coated with a mouse IgG1 monoclonal antibody (mAb Ber-EP4) specific for two (34 and 39 kDa) glycopolypeptide membrane antigens expressed on most normal and neoplastic human epithelial tissues. (Latza, U et al Ber-EP4: new monoclonal antibody which distinguishes epithelia from mesothelial, J. Clin Pathol. 43: 213-9, 1990.). The suspensions were then placed on a magnet, which attracts the beads and attached epithelial cells. The cells were then cultured in BCM, which is Ham's F-12 medium supplemented with 5% Foetal Calf Serum, epidermal growth factor, insulin, hydrocortisone, ethanolamine, apotransferrin, penicillin, streptomycin and amphoterecinb. (Gomm et al., 1997).

This protocol works well for metastatic lymph nodes but because the problem of ‘organoids’ does not arise, filtration is done using mesh >30 μm.

The technique of purifying normal breast cells is now well established and protocols modified from Gomm et al., 1995 were used.

Cytology and FISH

Cells from 15 tumours were cytospun on slides and stained with May Grunwald Giemsa (MGG) stain. The percentage of malignant cells was determined by conventional cytology before purification, after purification and after 7 days in culture following purification. FISH analysis of purified cells was performed as described (Kallioniemi, O. P et al ERBB2 amplification in breast cancer analyzed by fluorescence in situ hybridization, Proc Natl Acad Sci USA. 89: 5321-5, 1992.), using centromere-specific fluorescent probes (Vysis Inc.) for chromosomes 6, 7 and 12 (spectrum orange), 11, 17 and 18 (spectrum green) (Cajulis, R. S. and Frias-Hidvegi, D. Detection of numerical chromosomal abnormalities in malignant cells in fine needle aspirates by fluorescence in situ hybridization of interphase cell nuclei with chromosome-specific probes, Acta Cytol. 37: 391-6, 1993, Persons, D. L et al Chromosome-specific aneusomy in carcinoma of the breast, Clin Cancer Res. 2: 883-8, 1996.) were used to detect aneusomy. Cells were cytospun, denatured in formamide and then hybridised with the chromosome enumeration DNA probe (CEP DNA probe). Following hybridisation, the excess and unbound probe was removed by a series of washes and nuclei counterstained with DAPI (4,6 diamidino-2-phenylindole).

Immunostaining

ERα and progesterone receptor expression was assessed by immunohistochemistry on cell fixed in Zamboni reagent (Stefanini, M et al. Fixation of ejaculated spermatozoa for electron microscopy, Nature. 216: 173-4, 1967), using anti-ERα (MAb NC-ER-6F11 Novocastra, Newcastle, UK) and Progesterone receptor (PR) (MAbPR-88, Menarini Diagnostics, Wokingham, UK) according to manufacturers' protocols. Cell purity was determined by staining for cytokeratins 8 and 18 (MAb CAM 5.2 Becton Dickinson, Oxford, UK).

Proliferation and Viability

Cell viability was assessed using exclusion of Trypan blue staining. Proliferation of cells in culture was assessed by measuring tritiated thymidine (3HTdR) incorporation into DNA and using the MTS assay. For ³HTdR-labelling, purified cells were seeded into the wells of 24-well dishes. The medium was subsequently changed for 0.5 ml of ³HTdR-supplemented thymidine-free medium (RPMI, 2 mM L-glutamine, 100U/ml penicillin, 0.1 mg/ml streptomycin plus dialysed 2% FCS). The amount of incorporated ³HTdR in the cultures was measured by scintillation counting following precipitation with trichloroacetic acid. MTS assays were carried out using the Cell Titre 96 (Promega, UK), according to manufacturer's protocols.

Effects of Estradiol and Tamoxifen

1×10⁵ cells were plated into the wells of 96 well plates. Culture medium (modified BCM using phenol red free DME-F12+5% PR free double stripped serum) was changed on the third day for medium containing 17B-estradiol (E2; 10 nM) or 4-hydroxytamoxifen (QHT; 100 nM) or an equal volume of ethanol. Total and viable (Trypan blue) cell counts were measured during the time course of the experiment.

Results

Optimisation of Purification Procedure.

We have previously described procedures for the purification and culturing of normal human mammary luminal epithelial cells. In order to adapt these techniques to the purification and culturing of tumour cells we have evaluated the use of Ber-Ep4 in this context. We have been able to show that careful use of trypsinisation leads to single cell suspensions of tumour cells with normal cells remaining in aggregates large enough to be removed by conventional filtration. We also evaluated other parameters, including the ratio of beads to cells required to obtain tumour cell preparations of high purity. The optimised procedure is outlined in FIG. 6.

Characterisation of Isolated Tumour Cells.

Following purification the purity of the cells was initially assessed by cytology (FIG. 7) and immunohistochemistry for cytokeratin 8 and 18 expression (FIG. 8). On the basis of these analyses >95% of the purified cells showed cytokeratin 8 and 18 expression and abnormal cytology. We examined the purity of the first 15 preparations after optimisation of the procedure. The results indicated that there appeared to be only 1-5% contamination. This was mainly due to the presence of degenerate cells and occasional macrophages. The mean contamination was 2%. We carried out FISH on a subset of these using probes for 6 chromosomes and compared this with normal cells purified from a reduction mammoplasty specimen.

We compared the percentage showing aneusomy using paired chromosome probes before and after 7 days' culture, The results are shown in FIG. 9 and table 6. It can be seen that between 88-90% showed aneusomy. The figure for individual probes was 28-88%. In contrast, a very small proportion of normal breast cells showed aneusomy. Thus, of 5 preparations, between 6 and 9% of cells showed apparent abnormalities.

We examined the proportion of cells that retained viability and adherence in a subset of samples. FIG. 10 shows that, at day 3, more than 50% of cancer cells were viable and adhered to plastic. At day 9, this falls to approximately 40%. In contrast, the normal epithelial cells increased in number, so that the numbers at day 9 was greater than the number initially seeded. There was no evidence of proliferation of the cancer cells, despite the fact that the MTS assay indicates maintained cell viability. In addition, thymidine uptake measurements showed no uptake over the 16 days studied.

ERα and PR immunostaining on a subset of tumors over 9 days showed that these phenotypes were retained in the cells throughout the time course studied in vitro, although there appeared to be a slight reduction of PR (Table 7 and FIG. 11a) However, in 6 normal cultures studied, there was a substantial reduction in expression of ERα and PR within 24 hours of culture. ERα expression was not observed beyond this period whereas PR expression was lost in 4 cultures by 60 hours. (FIG. 11b) Cytokeratin staining revealed that between 95 and 100% of cells were positively stained.

Effects of Estradiol and Tamoxifen

We studied the effects of estradiol (E2) and 4-Hydroxy Tamoxifen (4OHT) in tumor and normal samples. Normal cells were studied at several time points whereas due to limited number of cells, studies were limited to 2 time points in tumors. Trypan Blue staining determined viable cell numbers. The medium was changed on the 3^{rd} day of culture to E2 and 4OHT-containing medium thereby allowing sufficient time for the cells to adhere to plastic.

Four out of the 7 tumors studied showed significant reduction in cell numbers after tamoxifen treatment compared to estradiol treatment and all were ER positive. ER negative tumors failed to show a response (e.g. FIG. 12)

TABLE 5
Patient Characteristics (n = 82)
Patient Characteristics. This shows the total number of patients
from whom tumors were obtained during the study.

| | Number | Percentage |
|---|---|---|
| Pathological size | | |
| <2 cm | 32 | 39 |
| >2 cm | 50 | 61 |
| ER Positive | 65 | 79 |
| PR Positive | 57 | 69 |
| Histology | | |
| Invasive Ductal | 61 | 74 |
| Other/Mixed Invasive | 21 | 26 |
| Nodal status | | |
| Positive | 42 | 51 |
| Negative | 40 | 49 |
| Grade | | |
| PD | 28 | 34 |
| MD | 45 | 55 |
| WD | 9 | 11 |

PD, MD & WD Poorly, moderately & well differentiated Tumors Inv Duc Invasice ductal carcinoma

Table 6

Percentage malignancy as determined by cytology before and after (Day 0) immunobead selection and after 7 days in culture and percentage aneusomy at Days 0 and 7 following purification as determined by fluorescent in situ hybridisation (FISH). Cell numbers after purification T2 2.8×10⁵, T3 3.6×10⁵, T4 2.2×10⁵, T5 1.2×⁵ and T6 4.5×10⁵

TABLE 6
Percentage aneusomy in malignant cells following purification

| | Chromosome | | | | | | Total | |
|---|---|---|---|---|---|---|---|---|
| Tumor | 6 | 7 | 11 | 12 | 17 | 18 | Aneusomy | Controls |
| T2 | 28 | 36 | 78 | 73 | 43 | 88 | 91 | 7 |
| T3 | N.D. | 88 | N.D. | 77 | 60 | 71 | 92 | 6 |
| T4 | 68 | 53 | 78 | 67 | 76 | 70 | 91 | 9 |
| T5 | 67 | 75 | 84 | 66 | 75 | 70 | 89 | 7 |
| T6 | 65 | 72 | 76 | N.D. | 55 | N.D. | 89 | 6 |

TABLE 7

| Tumour | Day 0 | | | Day 3 | | | Day 6 | | | Day 9 | | |
|---|---|---|---|---|---|---|---|---|---|---|---|---|
| (ERα/PR | Viable | | | Viable | | | Viable | | | Viable | | |
| status) | cells | ERα | PR | cells | ERα | PR | cells | ERα | PR | cells | ERα | PR |
| T1_{(+++/+)} | 0.7 | 35.0 | ND | 0.4 | 39.6 | ND | 0.3 | 37.1 | ND | 0.2 | 32.9 | ND |
| T4_{(+++/+++)} | 2.0 | 44.0 | ND | 1.5 | 27.3 | ND | 1.2 | 21.6 | ND | 1.1 | 24.1 | ND |
| T5_{(+++/+++)} | 0.9 | 28.4 | ND | 0.7 | 25.1 | ND | 0.6 | 25.4 | ND | 0.5 | 24.4 | ND |
| T6_{(+++/+)} | 4.1 | 65.0 | ND | 3.1 | 56.8 | ND | 2.6 | 71.2 | ND | 2.4 | 67.7 | ND |
| T7_{(+++/+++)} | 6.0 | 25.3 | 64.4 | 4.5 | 30.9 | 60.4 | 3.0 | 30.7 | 59.2 | 2.7 | 28.8 | 53.6 |
| T8_{(++/++)} | 26.6 | 79.5 | 81.5 | 21.6 | 80.2 | 67.2 | 14.9 | 70.2 | 62.2 | 13.5 | 60.1 | 64.0 |
| T9_{(-/-)} | 3.2 | 5.8 | 3 | 2.9 | 5.2 | <3 | 2.0 | 5.5 | <3 | ND | ND | ND |

### EXAMPLE 4

### Patient Treatment

A patient frequently presents with locally advanced breast cancer, where a masectomy would be usually indicated. Generally, a fine needle aspirate (FNA) is done to ascertain the diagnosis and to carry out an immunocytochemical stain for ER. However, since frequently the ER is non-functional, most clinicians give chemotherapy with cytotoxics to ensure a rapid response, despite the inevitable side effects. A FERA Test may now be performed instead. If the ER is functional and tamoxifen sensitive, tamoxifen may be given. If the ER is functional, but the reporter is only inhibited by oestrogen withdrawal, an aromatase inhibitor may be given. In our experience, 95% of all untreated breast carcinoma with a positive FERA would respond to either of these treatments.

Following regression and conservative surgery and radiation to remove the residual cancerous tissue, the patient is given 5 year treatment with the endocrine agent of choice.

For example, patients who present with a clinically palpable breast-lump have imaging (ultrasound or mammograms) when they present, and later (preferably on the same day) are seen by senior staff and informed of the results. If definitely cancer or suspicious, patients then have a “tru-cut” biopsy and may also have a “fine-needle-aspirate” (FNA). A week later they are seen with the result. At this time, if the cytology result is positive and the ER (or the tru-cut) is positive they are asked to consider receiving Arimidex (an aromatase inhibitor) by day (plus Goserelin/Prostap eg 3.75 mg in monthly or other LH2H agonist, if premenopausal) prior to surgery in one month. They are seen in the clinic at 2 or 4 weeks for tumour measurements.
Day 0: Imaging, core, FNA
   48 hr: Histology, ER, FERA
   Day 1: Start Arimidex (+optionally Goserelin)
   Day 30: Definitive surgery

Clinical response is determined by physical examination supplemented by mammography and ultrasonography. The measurement of disease is every two weeks for one month, following the WHO criteria for the evaluation of response. A key marker such as Ki67 may be used before and after the 1 month treatment: a reduction of that strongly suggests an antiproliferative effect of the antioestrogen drug.

After one month the patient has surgery as decided by the local multidisciplinary team (MDT). Postoperative treatment is as defined by standard protocol. However, if a clear response to neoadjuvant therapy has been observed, it is possible to continue Arimidex 1 mg/dg in postmenopausal patients. For premenopausal patients, recommended treatment may be continuing Goserelin 3.75 mg in monthly with Arimidex 1 mg daily, for a total of 2 years.

After relapsing (at 1-10 year after diagnosis) the FNA is repeated on relapsed tissue. The majority (95%) will still show a positive FERA test, but at this stage (Relapse 1) only 50% (approx) are sensitive to endocrine therapy; of the remainder, 10% are insensitive (needing chemotherapy) and 40% have reporter gene activity only inhibitable by EGFR or C-erb B2 anatagonists. 6-16 months after treatment of relapse 1 the disease regrows, and a further FERA is done (Relapse 2). After use of third-line treatment, relapse is frequently in pleura or ascites and the FERA test will indicate whether further endocrine and kinase inhibitor there is indicated. However, frequently chemotherapy is now required.

1-45. (canceled)

46. A method for providing information for deciding on a therapeutic strategy for a patient comprising a step of:
(a) assessing the activity of a transcription factor in a cell or cells from the patient, comprising the further steps of:
(1) exposing the cell or cells to a recombinant viral vector comprising a reporter gene which comprises a promoter under the control of the transription factor (transcription factor-dependent promoter); and
(2) assessing the expression of the reporter gene in said cell or cells.

47. A method as in claim 46 wherein the transcription factor is selected from the group consisting of NF-kB, ETS1, tumor suppressor gene p53, c-myc, a Forkhead transcription factor, E2F, AP1 and SRE transcription factors.

48. A method as in claim 46 wherein the transcription factor is a nuclear hormone receptor protein.

49. A method as in claim 48 wherein the transcription factor is an estrogen-dependent promoter.

50. A method for characterizing the hormone dependency of a primary cancer cell or cells comprising the steps of:
(a) exposing said primary cancer cell or cells to a recombinant viral vector comprising a reporter gene which comprises a hormone-dependent promoter; and
(b) assessing the expression of the reporter gene in said cell or cells.

51. A method as in either of claims 46 or 50 wherein the recombinant viral vector is selected from the group consisting of recombinant adenoviral vectors and lentiviral vectors.

52. A method as in claim 51 wherein the transcription factor or hormone-dependent promoter is an estrogen-dependent promoter.

53. A method as in claim 52 wherein the promoter comprises one or more Estrogen Response Elements (EREs).

54. A method as in claim 50 wherein the hormone dependent promoter is selected from the group consisting of androgen-dependent promoters, thyroid hormone dependent promoters, retinoic acid dependent promoters, progesterone-dependent promoters, PPRs (peroxisome proliferator receptors), and VDR (vitamin D receptor) dependent promoters.

55. A method as in claim 50 wherein the primary cancer cell is selected from the group consisting of primary breast cancer cells, endometrial cancer (EC) cells and ovarian cancer cells.

56. A method as in either of claims 46 or 50 wherein expression of the reporter gene is assessed using the polymerase chain reaction (PCR).

57. A method as in either of claims 46 or 50 wherein the expression of the reporter gene is assessed immunologically, using fluorescence measurements or by measuring enzymatic or other biological activity.

58. A method as in claim 57 wherein the reporter gene encodes luciferase or a polypeptide belonging to a green fluorescent protein (GFP) family of proteins.

59. A method as in either of claims 46 or 50 wherein said recombinant viral vector comprises one or more further reporter genes under the control of a promoter responsive to a signaling pathway involved in a mechanism selected from the group consisting of invasion, apoptosis and cell proliferation.

60. A method as in claim 59 wherein the promoter is responsive to one of the group consisting of NF-kB, ETS1, tumor suppressor gene p53, c-myc proto-oncogene, a Forkhead transcription factor, E2F, AP1 and SRE transcription factors.

61. A method as in claim 50 comprising the further steps of:
(c) determining a response of the reporter gene to exposure of the cell or cells to the hormone; and
(d) deciding on a treatment regime for a patient making use of the information on the reporter gene behavior.

62. A method as in claim 61 wherein the patient is one having a cancer selected from the group consisting of breast, endometrial and ovarian cancers; wherein the recombinant viral vector comprises an Estrogen Responsive Element (ERE)-controlled reporter gene; and wherein the hormone is estrogen.

63. A method as in claim 61 wherein cell or cells are exposed to a test compound selected from the group consisting of tamoxifen/4-OHT and Faslodex.

64. A method as in claim 61 wherein if the reporter gene is activated by tamoxifen, the treatment regime avoids administration of tamoxifen to the patient.

65. A method as in claim 64 wherein the treatment regime includes administration of a drug which lowers estrogen levels.

66. A method as in claim 62 wherein the Estrogen Responsive Element (ERE)-controlled reporter gene is active in the absence of estrogen or tamoxifen, the treatment regime includes administration of an epidermal growth factor receptor (EGFR) antagonist.

67. A method as in claim 62 wherein if the Estrogen Responsive Element (ERE)-reporter gene is inhibited to a greater extent by an antiestrogen than by tamoxifen, then the treatment regime includes administration of said antiestrogen to the patient.

68. A method as in either claim 61 or 62 wherein if the reporter gene is inactive in the presence of the hormone, then the treatment regime comprises surgery or chemotherapy.

69. A recombinant viral vector comprising a reporter gene which comprises a hormone-dependent promoter.

70. A recombinant viral vector as in claim 69 comprising a reporter gene which comprises a hormone-dependent promoter for use in a method for choosing a treatment regime according to claim 61.

71. A recombinant viral vector as in claim 70 comprising an Estrogen Responsive Element (ERE)-controlled reporter gene for use in a method for choosing a treatment regime for a patient according to claim 62.

72. A recombinant adenoviral vector comprising a reporter gene which comprises an Estrogen Responsive Element (ERE).

73. A recombinant adenoviral vector as in claim 72 wherein the reporter gene comprises a plurality of Estrogen Responsive Element (EREs).

74. A vector comprising a reporter gene selected from the group consisting of pAdTack vectors, pShuttle vectors, vectors derived from pAdtack vectors or pShuttle vectors for preparing a recombinant viral vector according to claim 69.

75. A recombinant viral vector as in either one of claims 69 or 72 wherein the viral vector does not comprise a reporter gene under the control of a constitutive promoter.

76. A recombinant viral vector according to any one of claims 69, 72 or 74 further comprising a pharmaceutically acceptable diluent or carrier.

77. A kit of parts comprising:
(a) a recombinant viral vector comprising a reporter gene which comprises a hormone-dependent promoter;
(b) a hormone selected from the group consisting of the hormone on which the hormone-dependent promoter is dependent and an analogue thereof which is able to promote transcription from the reporter; and
(c) optionally, also a material selected from the group consisting of an antagonist of the hormone receptor and a partial antagonist of the hormone receptor.

78. A kit of parts according to claim 77 comprising:
(a) a recombinant adenoviral vector comprising an estrogen-dependent reporter gene;
(b) estrogen; and
(c) optionally ingredients selected from the group consisting of Tamoxifen, Faslodex and other antiestrogens.

79. A kit of parts according to either of claims 78 or 79 further comprising a substrate for a reporter gene.
